(19) United States
(12) Patent Application Publication
(10) Pub. No.: US 2007/0059361 A1
(43) Pub. Date: Mar. 15, 2007

(54) FAST-DISINTEGRATING EPINEPHRINE TABLETS FOR BUCCAL OR SUBLINGUAL ADMINISTRATION

(75) Inventors: Mutasem Rawas-Qalaji, Winnipeg (CA); Keith Simons, Winnipeg (CA); Xiaochen Gu, Winnipeg (CA); Estelle Simons, Winnipeg (CA)

Correspondence Address: WILSON SONSINI GOODRICH & ROSATI, 650 PAGE MILL ROAD, PALO ALTO, CA, 94304-1050, US

(73) Assignee: University of Manitoba, Winnipeg (CA)

(21) Appl. No.: 11/530,360

(22) Filed: Sep. 8, 2006

Related U.S. Application Data
(60) Provisional application No. 60/715,180, filed on Sep. 9, 2005.
(60) Provisional application No. 60/759,039, filed on Jan. 17, 2006.

(51) Int. Cl. A61K 9/20; A61K 31/137

(52) U.S. Cl. 424/464; 514/649

(57) ABSTRACT

Described herein are formulations for fast-disintegrating epinephrine tablets which can be prepared for buccal or sublingual administration, wherein the fast-disintegrating epinephrine tablets can produce plasma epinephrine concentrations similar to those achieved by an approximately 0.3 mg epinephrine dose in the thigh (Epi-Pen).

### RELATED APPLICATIONS

This application claims the benefit of U.S. Provisional Application No. 60/715,180, filed Sep. 9, 2005, and U.S. Provisional Application No. 60/759,039, filed Jan. 16, 2006, which are hereby incorporated by reference in their entireties.

### FIELD OF THE INVENTION

Described herein are formulations for fast-disintegrating epinephrine tablets which can be prepared for buccal or sublingual administration, wherein the fast-disintegrating epinephrine tablets can produce plasma epinephrine concentrations similar to those achieved by an approximately 0.3 mg epinephrine dose in the thigh (Epi-Pen).

### BACKGROUND OF THE INVENTION

Tablets that disintegrate or dissolve rapidly in the patient's mouth without the use of water are convenient for the elderly, young children, patients with swallowing difficulties, and in situations where water is not available. For these specially designed formulations, the small volume of saliva that is available is sufficient to disintegrate or dissolve a tablet in the oral cavity. The drug released from these tablets can be absorbed partially or entirely into the systemic circulation from the buccal mucosa or sublingual cavity, or can be swallowed as a solution to be absorbed from the gastrointestinal tract.

The sublingual route usually produces a faster onset of action than traditional orally administered tablets and the portion absorbed through the sublingual blood vessels bypasses the hepatic first pass metabolic processes (Birudaraj et al., 2004, J Pharm Sci 94; Motwani et al., 1991, Clin Pharmacokinet 21: 83-94; Ishikawa et al., 2001, Chem Pharm Bull 49: 230-232; Price et al., 1997, Obstet Gynecol 89: 340-345; Kroboth et al., 1995, J Clin Psychopharmacol 15: 259-262; Cunningham et al., 1994, J Clin Anesth 6: 430-433; Scavone et al., 1992, Eur J Clin Pharmacol 42: 439-443; Spenard et al., 1988, Biopharm Drug Dispos 9: 457-464).

Likewise, due to high buccal vascularity, buccally delivered drugs can gain direct access to the systemic circulation and are not subject to first-pass hepatic metabolism. In addition, therapeutic agents administered via the buccal route are not exposed to the acidic environment of the gastrointestinal tract (Mitra et al., 2002, Encyclopedia of Pharm. Tech., 2081-2095). Further, the buccal mucosa has low enzymatic activity relative to the nasal and rectal routes. Thus, the potential for drug inactivation due to biochemical degradation is less rapid and extensive than other administration routes (de Varies et al., 1991, Crit. Rev. Ther. Drug Carr. Syst. 8: 271-303).

The buccal mucosa is also highly accessible, which allows for the use of tablets which are painless, easily administered, easily removed, and easily targeted. Because the oral cavity consists of a pair of buccal mucosa, tablets, such as fast disintegrating tablets, can be applied at various sites either on the same mucosa or, alternatively, on the left or right buccal mucosa (Mitra et al., 2002, Encyclopedia of Pharm. Tech., 2081-2095). In addition, the buccal route could be useful for drug administration to unconscious patients, patients undergoing an anaphylactic attack, or patients who sense the onset of an anaphylactic attack.

Epinephrine (EP) is the drug of choice for the treatment of anaphylaxis worldwide (Joint Task Force on Practice Parameters, 2005, J Allergy Clin Immunol 115: S483-S523; Lieberman, 2003, Curr Opin Allergy Clin Immunol 3: 313-318; Simons, 2004, J Allergy Clin Immunol 113: 837-844). It is available only as an injectable dosage form in ampoules or in autoinjectors. In aqueous solutions, epinephrine is unstable in the presence of light, oxygen, heat, and neutral or alkaline pH values (Connors et al., 1986, in Chemical Stability of Pharmaceuticals: A Handbook for Pharmacists, Wiley-Interscience Publication: New York). Feasibility studies in humans and animals have shown that EP can be absorbed sublingually (Gu et al., 2002, Biopharm Drug Dispos 23: 213-216; Simons et al., 2004, J Allergy Clin Immunol 113: 425-438). The recommended dose of EP for the treatment of anaphylaxis is about 0.01 mg/Kg: usually about 0.2 mL to about 0.5 mL of a 1:1000 dilution of EP in a suitable carrier. Based on historical and anecdotal evidence, an approximately 0.3 mg dose of EP, by subcutaneous (SC) or intramuscular (IM) injection into the deltoid muscle, has been agreed upon as the dose required for the emergency treatment of anaphylaxis. Recent studies have demonstrated that if the approximately 0.3 mg dose is administered IM into the laterus vascularis (thigh) muscle, EP plasma concentrations are higher and occur more quickly than SC or IM administration into the deltoid muscle. (Joint Task Force on Practice Parameters, 2005, J Allergy Clin Immunol 115: S483-S523; Lieberman, 2003, Curr Opin Allergy Clin Immunol 3: 313-318; Simons, 2004, J Allergy Clin Immunol 113: 837-844)).

As stated above, EP is typically administered either subcutaneously or intramuscularly by injection. Thus, EP injections are the accepted first aid means of delivering EP and are administered either manually or by automatic injectors. It is recommended that persons at risk of anaphylaxis, and persons responsible for children at risk for anaphylaxis, maintain one or more automatic EP injectors in a convenient place at all times.

Given the difficulties associated with manual subcutaneous or intramuscular administration of EP, such as patient apprehension related to injections or the burden of an at risk person having to always maintain an EP injector close at hand, there exists a need in the art for more convenient dosage forms which can provide immediate administration of EP to a person undergoing anaphylaxis wherein the need for injection or EP injectors is obviated.

We hypothesized that EP could be formulated into a fast disintegrating buccal or sublingual tablet (e.g., oral disintegrating tablets (ODTs)) containing a suitable dose that would result in plasma EP concentrations similar to those produced by the recommended intramuscular dose of approximately 0.3 mg of EP for adults, by selecting the appropriate pharmaceutical excipients in the right proportions, in combination with optimal manufacturing techniques and compression parameters. Our aim in this study was to systemically evaluate the effect of incorporating increasing loads of EP as epinephrine bitartrate (EPBT) on the hardness, disintegration time, and wetting time of sublingual tablet formulations containing a super disintegrant in order to develop a tablet that contained sufficient EPBT that when administered sublingually would result in epinephrine plasma concentrations similar to those achieved following the intramuscular injection of 0.3 mg EP into the thigh muscle.

### SUMMARY OF THE INVENTION

According to a one aspect of the invention, there is provided a pharmaceutical tablet for buccal or sublingual application comprising: about 48.5% epinephrine (EPBT); about 44.5% microcrystalline cellulose; about 5% low-substituted hydroxypropyl cellulose; and about 2% Magnesium stearate.

According to another aspect of the invention, there is provided a pharmaceutical tablet for buccal or sublingual application comprising: about 72.8 mg epinephrine (EPBT); about 66.8 mg microcrystalline cellulose; about 7.4 mg low-substituted hydroxypropyl cellulose; and about 3 mg Magnesium stearate.

According to yet another aspect of the invention, there is provided a method of preparing an epinephrine tablet for buccal or sublingual administration comprising: preparing a mixture of: about 48.5% epinephrine (EPBT); about 44.5% microcrystalline cellulose; about 5% low-substituted hydroxypropyl cellulose; and about 2% Magnesium stearate; and compressing unit dosage portions of the mixture to about 24 kN, thereby producing a tablet.

In still another aspect of the invention, there is provided a pharmaceutical tablet for buccal or sublingual application comprising: about 24.26% epinephrine (EPBT); about 66.37% microcrystalline cellulose; about 7.37% low-substituted hydroxypropyl cellulose; and about 2% Magnesium stearate.

In a further aspect of the invention, there is provided a pharmaceutical tablet for buccal or sublingual application comprising: about 36.4 mg epinephrine (EPBT); about 99.5 mg microcrystalline cellulose; about 11.1 mg low-substituted hydroxypropyl cellulose; and about 3 mg Magnesium stearate.

In still other aspects of the invention, there are provided pharmaceutical tablets for buccal or sublingual administration comprising: about 0.5% to about 90% epinephrine; about 7.5% to about 95% filler; and about 2.5% to about 10.5% disintegrant. In certain embodiments, the buccal or sublingual tablet comprises about 35% to about 85% epinephrine. In other embodiments, the buccal or sublingual tablet comprises about 40% to about 70% epinephrine. In still other embodiments, the buccal or sublingual tablet comprises about 40% to about 55% epinephrine. In yet other embodiments, the buccal or sublingual tablet comprises about 65% to about 90% epinephrine. In one embodiment, the buccal or sublingual tablet comprises about 35% to about 45% epinephrine. In another embodiment, the buccal or sublingual tablet comprises about 20% to about 35% epinephrine. In still another embodiment, the buccal or sublingual tablet comprises about 10% to about 15% epinephrine. In yet another embodiment, the buccal or sublingual tablet comprises about 2% to about 8% epinephrine.

In certain aspects of the invention, there are provided pharmaceutical tablets for buccal or sublingual tablet administration comprising about 25 mg to about 75 mg of epinephrine. In certain embodiments, the buccal or sublingual tablet comprises about 35 mg to about 60 mg of epinephrine. In other embodiments, the buccal or sublingual tablet comprises about 35 mg to about 45 mg of epinephrine. In still other embodiments, the buccal or sublingual tablet comprises about 55 mg to about 75 mg of epinephrine. In one embodiment, the buccal or sublingual tablet comprises about 25 mg to about 40 mg of epinephrine. In another embodiment, the buccal or sublingual tablet comprises about 10 mg to about 25 mg of epinephrine. In yet another embodiment, the buccal or sublingual tablet comprises about 5 mg to about 10 mg of epinephrine. In still another embodiment, the buccal or sublingual tablet comprises about 0.5 mg to about 5 mg of epinephrine.

In other aspects of the present invention, the epinephrine is selected from the group consisting of: racemic mixtures of epinephrine, free base epinephrine, epinephrine bitartrate (EPBT), or epinephrine HCl.

In still other aspects of the present invention, the filler can be selected from the group consisting of: microcrystalline cellulose having a particle size range of about 5 μm to about 500 μm, lactose, calcium carbonate, calcium bicarbonate, calcium phosphate, dibasic calcium phosphate, calcium sulfate, calcium silicate, cellulose powders, dextrose, dextrates, dextrans, starches, pregelatinized starches, sucrose, xylitol, lactitol, sorbitol, sodium bicarbonate, sodium chloride, polyethylene glycol, or combinations thereof.

In yet other aspects of the present invention, the disintegrant can be selected from the group consisting of: low-substituted hydroxypropyl celluloses, cross-linked celluloses, cross-linked sodium carboxymethyl celluloses, cross-linked carboxymethyl celluloses, cross-linked croscarmelloses, cross-linked starches, sodium starch glycolate, crospovidone, or combinations thereof.

In other embodiments, the pharmaceutical tablet for buccal or sublingual administration comprising epinephrine can further comprise a pharmaceutically acceptable excipient. In certain embodiments, the pharmaceutically acceptable excipient is selected from the group consisting of: diluents, binders, glidants, lubricants, colorants, flavorants, coating materials, or combinations thereof.

In yet other embodiments, the invention described herein provides a pharmaceutical tablet comprising epinephrine having long term stability. In certain embodiments, the pharmaceutical tablet displays a decrease in the content of epinephrine after being stored at 25° C. for at least twelve months of less than 2.5 percent. In other embodiments, the pharmaceutical tablet displays a decrease in the content of epinephrine after being stored at 5° C. for at least twelve months of less than 2.5 percent. In still other embodiments, the pharmaceutical tablet displays a decrease in the content of epinephrine after being stored at 5° C. with nitrogen flushing for at least twelve months of less than 2.5 percent. In certain embodiments, the pharmaceutical tablet comprises from about 10 mg to about 40 mg of epinephrine.

In other embodiments, the invention described herein provides a method of preparing an epinephrine tablet for sublingual administration comprising preparing a mixture of: about 0.5% to about 90% epinephrine; (b) about 7.5% to about 95% filler; about 2.5% to about 10.5% disintegrant; and compressing unit dosage portions of the mixture to about 24 kN, thereby producing a tablet.

In still other embodiments, a method is provided for the treatment of an allergic emergency, comprising the administration of a dose a pharmaceutical tablet for buccal or sublingual described herein to a person diagnosed with, or suspected of having, an allergic emergency. In one embodiment, the allergic emergency is anaphylaxis. In another embodiment, the allergic emergency is asthma. In still another embodiment, the allergic emergency is bronchial asthma.

In certain other embodiments, a method is provided for the treatment of a cardiac event, comprising the administration of a dose of a pharmaceutical tablet for buccal or sublingual described herein to a patient diagnosed with, or suspected of having, a cardiac event. In one embodiment, the cardiac event is a cardiac arrest.

### BRIEF DESCRIPTION OF THE DRAWINGS

FIG. 1: Effect of increasing compression force on tablet hardness. All formulations showed an exponential increase in tablet hardness (Equation I) upon a linear increase in the compression force. R² is ≧0.98 in all formulations. Each data point is expressed as the mean±SD of 5 measurements from the same batch.

FIG. 2: Effect of increasing compression force on tablet disintegration time. Formulation A (0 mg EP) (excluding data at 24 Kgf) showed a linear increase in disintegration time (Equation II), while formulations B (5 mg EP) (excluding data at 25 Kgf), C (10 mg EP) and D (20 mg EP) showed an exponential increase in the disintegration time (Equation I). R² is ≧0.91 in all formulations. Each data point is expressed as the mean±SD of 5 measurements from the same batch.

FIG. 3: Effect of increasing compression force on tablet wetting time. Formulation A (excluding data at 24 Kgf) showed a linear increase in wetting time (Equation II), while formulations B, C, and D showed an exponential increase in the wetting time (Equation I). R² is ≧0.92 in all formulations. Each data point is expressed as the mean±SD of 5 measurements from the same batch.

FIG. 4: Relationship between tablet hardness and disintegration time. Each data point is expressed as the mean±SD of 5 measurements from the same batch.

FIG. 5: Relationship between tablet hardness and wetting time. Each data point is expressed as the mean±SD of 5 measurements from the same batch.

FIG. 6: Correlation between tablet disintegration and wetting time. R² is ≧0.92 in all formulations. Each data point is expressed as the mean±SD of 5 measurements from the same batch.

FIG. 7: Plasma epinephrine concentration of four doses of Formulation I: I-A (0 mg EP), I-B (10 mg EP), I-C (20 mg EP), I-D (40 mg EP) versus time plots after administration of the epinephrine sublingually and after epinephrine IM injection.

FIG. 8: Plasma epinephrine concentration versus time plots after administration of epinephrine sublingually of four different tablet formulations (Formulations I-D, II-E, III-F and IV-G as set forth in Table XII) and after epinephrine IM injection (EpiPen). Mean (±SEM) AUC, Cₘₐₓ, and Tₘₐₓ after administration of 40 mg epinephrine sublingual tablets of formulation I-D and EpiPen IM injections (n=5) were not significantly different (p>0.05).

FIG. 9: Microscopic pictures of the dissolution of EPBT crystals in water over 3 min.

FIG. 10: Microscopic pictures of the dissolution of EPBT crystals in a saturated solution of mannitol over 5 min.

### INCORPORATION BY REFERENCE

All publications and patent applications mentioned in this specification are herein incorporated by reference to the same extent as it each individual publication or patent application was specifically and individually indicated to be incorporated by reference.

### DETAILED DESCRIPTION OF THE INVENTION

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described.

As described herein, buccal or sublingual oral disintegrating tablets (ODTs) are distinguished from conventional sublingual tablets, lozenges, or buccal tablets by the ODTs' ability to fully dissolve or disintegrate in less than about one minute in the mouth.

Methods of Manufacturing Buccal and Sublingual Disintegrating Tablets Comprising Epinephrine

Manufacturing processes for buccal and sublingual disintegrating tablets are known in the art and include, but are not limited to, conventional tableting techniques, freeze-dried technology, and floss-based tableting technology.

i. Conventional Tableting Techniques

Conventional tablet processing features conventional tablet characteristics for ease of handling, packaging, and fast disintegration (T. K. Ghosh, Oct. 29, 2003, American Association of Pharmaceutical Scientists). The technology is based on a combination of physically modified polysaccharides that have water dissolution characteristics that facilitate fast disintegration and high compressibility. The result is a fast-disintegrating tablet that has adequate hardness for packaging in bottles and easy handling.

In certain embodiments, the manufacturing process involves granulating low-moldable sugars (e.g., mannitol, lactose, glucose, sucrose, and erythritol) that show quick dissolution characteristics with high-moldable sugars (e.g., maltose, sorbitol, trehalose, and maltitol). The result is a mixture of excipients that have fast-dissolving and highly moldable characteristics (Hamilton et al., 2005, Drug Deliv. Technol. 5: 34-37). The epinephrine can be added, along with other standard tableting excipients, during the granulation or blending processes. The tablets are manufactured at a low compression force followed by an optional humidity conditioning treatment to increase tablet hardness (Parakh et al., 2003, Pharm. Tech. 27: 92-100).

In other embodiments, a compressed buccal or sublingual tablet comprising epinephrine is based on a conventional tableting process involving the direct compression of active ingredients, effervescent excipients, and taste-masking agents (see U.S. Pat. No. 5,223,614, which is herein incorporated by reference in its entirety). The tablet quickly disintegrates because effervescent carbon dioxide is produced upon contact with moisture. The effervescent excipient (known as effervescence couple) is prepared by coating the organic acid crystals using a stoichiometrically lesser amount of base material. The particle size of the organic acid crystals is carefully chosen to be larger than the base excipient to ensure uniform coating of the base excipient onto the acid crystals. The coating process is initiated by the addition of a reaction initiator, which is purified water in this case. The reaction is allowed to proceed only to the extent of completing the base coating on organic acid crystals. The required end-point for reaction termination is determined by measuring carbon dioxide evolution. Then, the excipient is mixed with the active ingredient or active microparticles and with other standard tableting excipients and then compressed into tablets.

In still other embodiments, the buccal or sublingual tablets are made by combining non-compressible fillers with a taste-masking excipient and active ingredient into a dry blend. The blend is compressed into tablets using a conventional rotary tablet press. Tablets made with this process have higher mechanical strength and are sufficiently robust to be packaged in blister packs or bottles (Aurora et al., 2005, Drug Deliv. Technol. 5:50-54). In other embodiments, the method further incorporates taste-masking sweeteners and flavoring agents such as mint, cherry, and orange. In certain embodiments, epinephrine tablets made with this process should disintegrate in the mouth in 5-45 seconds and can be formulated to be bioequivalent to intramuscular or subcutaneous dosage forms containing epinephrine.

ii. Freeze-Dried Buccal or Sublingual Tablets Comprising Epinephrine

The freeze-drying process involves the removal of water (by sublimation upon freeze drying) from the liquid mixture of a drug (e.g., epinephrine), matrix former, and other excipients filled into preformed blister pockets. The formed matrix structure is very porous in nature and rapidly dissolves or disintegrates upon contact with saliva (Sastry el a., 2005, Drug Delivery to the Oral Cavity: Molecule to Market, pp. 311-316).

Common matrix-forming agents include gelatins, dextrans, or alginates which form glassy amorphous mixtures for providing structural strength; saccharides such as mannitol or sorbitol for imparting crystallinity and hardness; and water, which functions as a manufacturing process medium during the freeze-drying step to induce the porous structure upon sublimation. In addition, the matrix may contain taste-masking agents such as sweeteners, flavorants, pH-adjusting agents such as citric acid, and preservatives to ensure the aqueous stability of the suspended drug in media before sublimation.

In this embodiment, Freeze-dried buccal or sublingual ODTs comprising epinephrine can be manufactured and packaged in polyvinyl chloride or polyvinylidene chloride plastic packs, or they may be packed into laminates or aluminum multilaminate foil pouches to protect the product from external moisture.

Other known methods for manufacturing buccal or sublingual ODTs include lyophilization (e.g., Lyoc (Farmalyoc, now Cephalon, Franzer, Pa.) and QuickSolv (Janssen Pharmaceutica, Beerse, Belgium). Lyoc is a porous, solid wafer manufactured by lyophilizing an oil-in-water emulsion placed directly in a blister and subsequently sealed. The wafer can accommodate high drug dosing and disintegrates rapidly but has poor mechanical strength (see EP 0159237). QuickSolv tablets are made with a similar technology that creates a porous solid matrix by freezing an aqueous dispersion or solution of the matrix formulation. The process works by removing water using an excess of alcohol (solvent extraction). In certain embodiments, the manufacturing methods which utilize the lyophilization techniques, such as those related to QuickSolv as described above, could be of particular importance for producing buccal or sublingual ODTs comprising epinephrine. This is especially so in light of the data provided herein which shows the potential negative effect that highly water soluble excipients can have in the absorption of epinephrine in vivo. Thus, a buccal or sublingual ODT comprising epinephrine manufactured by such a lyophilization technique could provide increased in vivo epinephrine absorption due of the removal of water soluble excipients occurring during the water removal step as described above.

iii Floss-Based Buccal or Sublingual Tablets Comprising Epinephrine

In other embodiments, floss-based tablet technology (e.g., FlashDose, Biovail, Mississauga, ON, Canada) can be used to produce fast-dissolving buccal or sublingual tablets comprising epinephrine using a floss known as the shearform matrix. This floss is commonly composed of saccharides such as sucrose, dextrose, lactose, and fructose. The saccharides are converted into floss by the simultaneous action of flash-melting and centrifugal force in a heat-processing machine similar to that used to make cotton candy. See U.S. Pat. Nos. 5,587,172, 5,622,717, 5,567,439, 5,871,781, 5,654,003, and 5,622,716, each of which is specifically incorporated by reference herein in their entirety. The fibers produced are usually amorphous in nature and are partially re-crystallized, which results in a free-flowing floss. The floss can be mixed with epinephrine and pharmaceutically acceptable excipients followed by compression into a tablet that has fast-dissolving characteristics.

iv. Additional Method of Formulating Buccal or Sublingual Tablets Comprising Epinephrine

Additional techniques can also be used to formulate the rapidly disintegrating or dissolving buccal or sublingual tablets of the present invention (Sastry et al., 2000, Pharm Sci Technol Today 3: 138-145; Chang et al., 2000, Pharmaceutical Technology 24: 52-58; Sharma et al., 2003, Pharmaceutical Technology North America 10-15; Allen, 2003, International Journal of Pharmaceutical Technology 7: 449-450; Dobetti, 2000, Pharmaceutical Technology Europe 12: 32-42; Verma and Garg, 2001, Pharmaceutical Technology On-Line 25: 1-14). Direct compression, one of these techniques, requires the incorporation of a super disintegrant into the formulation, or the use of highly water soluble excipients to achieve fast tablet disintegration or dissolution. Direct compression does not require the use of moisture or heat during tablet formation process, so it is very useful for the formulation and compression of tablets containing moisture-labile and heat-labile medications. However, the direct compression method is very sensitive to changes in the types and proportions of excipients, and in the compression force (CF), when used to achieve tablets of suitable hardness without compromising the rapid disintegration capabilities. As will be appreciated by one of skill in the art, in order for tablets administered sublingually to release the dose of medication for maximum rate and extent of absorption, the tablet must disintegrate almost instantaneously following insertion into the sublingual cavity. Precise selection and evaluation of the type and proportion of excipients used to formulate the tablet control the extent of hardness and rate of disintegration. Compression force (CF) can also be adjusted to result in tablets that have lower hardness (H) and disintegrate more quickly. Unique packaging methods such as strip packaging may be required to compensate for the problem of extreme friability of rapidly disintegrating, direct compression tablets.

Watenabe et al. (Watanabe et al., 1995, Biol Pharm Bull 18: 1308-1310; Ishikawa et al., 2001, Chem Pharm Bull 49: 134-139) and Bi et al (Bi et al., 1996, Chem Pharm Bull 44: 2121-2127; Bi et al., 1999, Drug Dev Ind Pharm 25: 571-581) were the first to evaluate the ideal excipient proportions and other related parameters required to formulate durable fast disintegrating tablets using a super disintegrant. They studied the effect of a wide range of microcrystalline cellulose: low-substituted hydroxypropyl cellulose (MCC:L HPC) ratios on the tablet characteristics. A ratio of 9:1 and 8:2 resulted in greater tablet hardness in association with faster disintegration and wetting times. Similar results were reported by Bi et al (Bi et al., 1996; Bi et al., 1999). Based on the results obtained by Watanabe et al and Bi et al, we selected a MCC:L HPC ratio of 9:1 as the optimal ratio to test our preliminary sublingual epinephrine tablet formulations.

Formulations of fast-disintegrating epinephrine tablets for buccal or sublingual administration at increasing epinephrine loads (e.g., 0%, 12%, 24% and 48%) are feasible. As will be appreciated by one of skill in the art, any suitable form of epinephrine, for example, a racemic mixture of epinephrine isomers, the free base form of epinephrine, as well as any suitable pharmaceutical salt can be used within the invention. In a preferred embodiment, epinephrine bitartrate or epinephrine HCl salt could be used provided that the epinephrine is in a form suitable for incorporation into the buccal or sublingual tablet and the epinephrine is primarily in the “active” isomer, that is, for example, greater than 50%, greater than 60%, greater than 70%, greater than 80%, greater than 85%, greater than 90%, greater than 92%, greater than 94%, greater than 95%, greater than 96%, greater than 97%, greater than 98%, or greater than 99% of the epinephrine pharmaceutically-acceptable salt is in the active isomer form. Epinephrine when synthesized occurs as a racemic mixture comprised of 50% as the L-epinephrine isomer and 50% as the D-epinephrine isomer. Only the L-epinephrine isomer is physiologically and pharmacologically active in the mammalian body. Following synthesis of the racemic mixture, the epinephrine is exposed to D-tartaric acid and the L-epinephrine crystallizes out as the L-epinephrine-D-bitartrate salt.

Maintaining tablet hardness at the lower range would result in rapid disintegration times (DT) and short wetting times (WT) despite the increase in epinephrine load. The tablets demonstrated fast disintegration (<10 sec) and wetting (<30 sec) times. Disintegration and wetting tests were performed in this study using simple and rapid techniques that resemble the parameters and the conditions in the sublingual area in humans and animals. These tablets had sufficient H (3-4 Kgf) to withstand shipping and handling. If the tablet either contains the improper excipients, or the correct excipients in the improper proportions, or if it is compressed by too much force, then it will not disintegrate rapidly. If the tablet is formulated with the correct excipients in the correct proportions but compressed with insufficient force, then the tablets readily break apart and could disintegrate totally into powder with usual shipping or handling stress, and would be useless for patient administration. A further increase in the drug load might be possible at the expense of tablet hardness that could necessitate special packaging of the tablets. This could involve individual packages for each tablet, or strip or unit-dose packaging. This approach is often used in hospitals and there are a number of commercially available formulations that use this approach such as acetaminophen, “fast-melt” or Alka-Seltzer Tablets.

### DETAILED DESCRIPTION OF PREFERRED EMBODIMENTS

In certain aspects of the present invention, we have determined herein that by using formulation I-D, as set forth in Table VI, containing a dose of 40 mg EP as the EPBT salt, and administering the tablet sublingually, that plasma epinephrine concentrations similar to those achieved by the 0.3 mg epinephrine dose in the thigh (Epi-Pen) can be demonstrated using the rabbit model. However, as will be known by one of skill in the art, the correct epinephrine plasma concentrations for the emergency treatment of anaphylaxis are unknown, although the 0.3 mg IM dose is effective and prevents death from anaphylaxis. Since this is the “gold” standard, the epinephrine sublingual administration should be able to achieve plasma epinephrine concentrations of a “similar order”.

Four sublingual tablet formulations that contain the same excipients composition (Formulation I) and a series of increasing doses of epinephrine (I-A, I-B, I-C, and I-D) were prepared by a direct compression method and are summarized in Table VI. Excipients in formulations I (I-A, I-B, I-C, and I-D) were selected to provide fast tablet disintegration. The results of the in vitro testing of each formulation are shown in Table X.

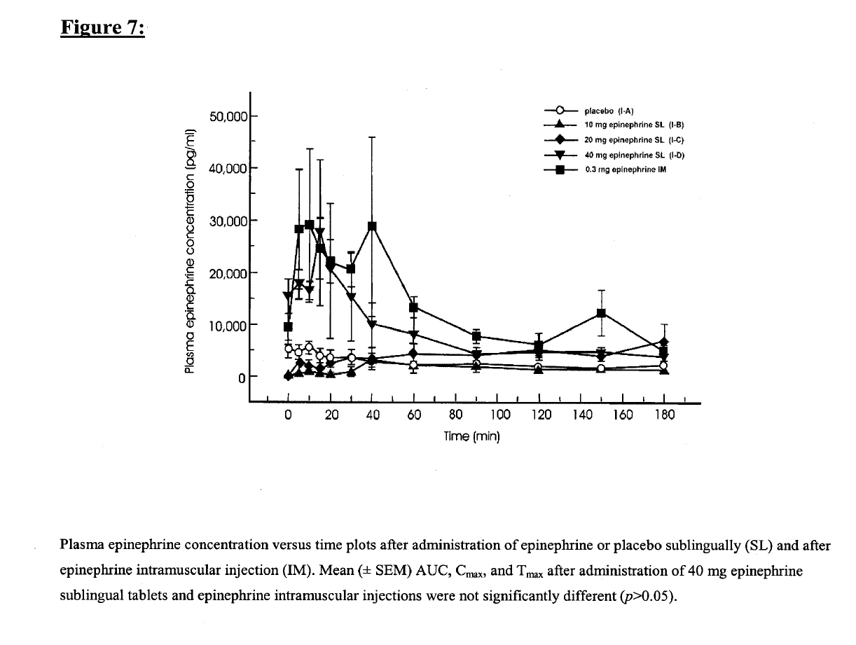

In certain other aspects of the present invention, sublingual tablet formulations of the present invention can be tested in in vivo models. In one embodiment, sublingual tablet formulations I (I-A, I-B, I-C, and I-D) were tested in an in vivo rabbit model, as set forth in the IN VIVO METHODS described below, to determine the plasma epinephrine concentrations in comparison to a 0.3 mg epinephrine intramuscular (IM) dose in the thigh muscle (EpiPen). A 0.3 mg of dose epinephrine IM in the thigh, either by syringe or autoinjector (EpiPen) is the recommended treatment of anaphylaxis in adults. As shown in the examples that follow, formulation I-D, which contains 40 mg of epinephrine, resulted in plasma epinephrine concentrations (area under the curve) not significantly different from the concentrations obtained following a 0.3 mg EpiPen intramuscular injection in the thigh of a rabbit. The results of the in vivo testing for Formulations I-A, I-B, I-C, and I-D in comparison to EpiPen® are shown in FIG. 7 and Table XI.

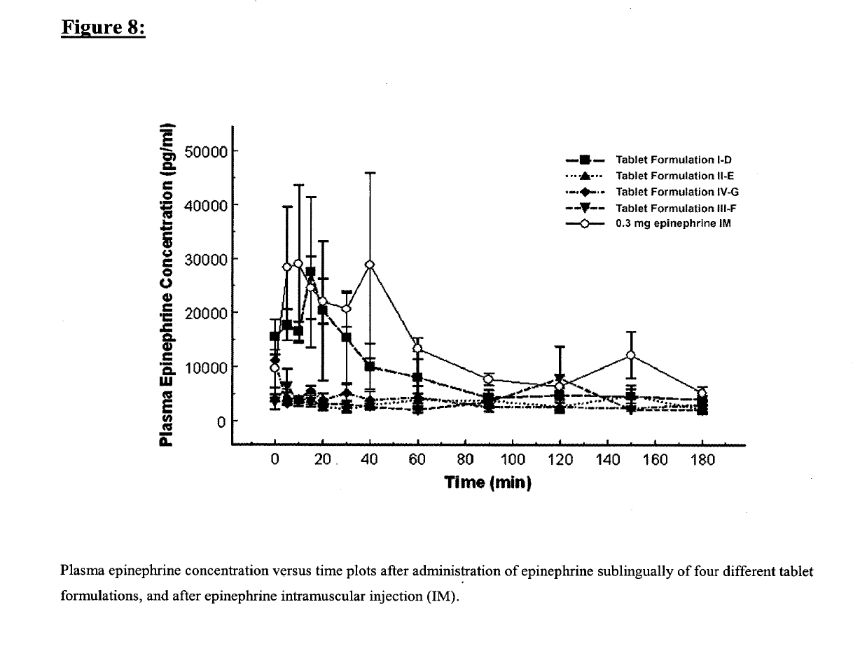

To confirm the unique characteristics of Formulation I-D, three (3) additional sublingual epinephrine tablet formulations, II (II-E, Table VII), III (III-F, Table VIII), IV (IV-G, Table IX), that also contain 40 mg of epinephrine were prepared by using other types of excipients in various proportions. Excipients in formulations II-E, III-F, and IV-G were selected to provide fast tablet disintegration. The results of the in vitro testing of each formulation are shown in Table X. As can be seen in FIG. 8, formulations II-E, III-F, and IV-G resulted in plasma epinephrine concentrations (area under the curve) significantly lower than the concentrations obtained following a 0.3 mg EpiPen intramuscular injection, and following the sublingual administration of Formulation I-D in a rabbit.

While not wishing to be bound to a specific hypothesis, the inventors believe that I-B and I-C did not result in epinephrine plasma concentrations similar to those achieved with 0.3 mg IM because there was insufficient epinephrine in I-B and I-C available to be absorbed rapidly by the sublingual route. However, these formulations could be useful for infants and children where lower doses may be given by SC or IM injection. For example, a 0.01 mg/kg dose by injection for a 9 kg child would require a 0.09 mg dose of epinephrine if injected. This 0.09 mg dose IM in the thigh could be compared with the 10 mg EP or 20 mg EP sublingual tablets.

Again, while not wishing to be bound to a particular hypothesis, the inventors believe that the excipients in II-E, III-F and IV-G in some way inhibited the dissolution of epinephrine from the tablet so that it is not available for absorption by the sublingual route. As will be appreciated by one of skill in the art, it may be possible to prepare other formulations with other excipients with varying proportions and using various compression forces that result in hardness (H) and disintegration times (DT) that result in the release of epinephrine from the tablets quickly enough to be absorbed sublingually. However, II-E, III-F and IV-G results show that even if H, DT and WT are similar in the in vitro quality control testing (Table X), the formulations may not work in vivo (Table XI).

Thus, the type, proportion, and even the grade and solubility of the excipients are important. We have shown that at least three (3) other formulations, II-E, III-F and IV-G also containing 40 mg epinephrine like I-D, do not work. As will be apparent to a person of skill in the art, in the case where substantial amounts of highly water-soluble excipients (e.g., mannitol) reduce the dissolution of epinephrine by saturating the available solution in the sublingual cavity, although the tablet disintegrates, the epinephrine is not available for sublingual absorption.

Thus, in summary, in order for tablets administered buccal or sublingually to release the dose of medication for maximum rate and extent of absorption, the tablets must disintegrate almost instantaneously following insertion into the sublingual cavity. Therefore, selection and evaluation of the type and proportion of excipients used to formulate the tablet control the extent of hardness and rate of disintegration is required. The value of the non-medicinal ingredients and their ratios is fully demonstrated by the inability of Formulations II-E, III-F and IV-G 40 mg EP sublingual tablets to achieve epinephrine plasma concentrations similar to the 0.3 mg Epi-pen IM dose and Formulation I-D, 40 mg EP sublingual dose.

i. Fast-Disintegrating Buccal or Sublingual Tablets Comprising Epinephrine

According to another embodiment of the invention, there is provided a pharmaceutical tablet for buccal or sublingual application comprising: about 48.5% epinephrine (EPBT); about 44.5% microcrystalline cellulose; about 5% low-substituted hydroxypropyl cellulose; and about 2% Magnesium stearate.

In another aspect of the invention, there is provided a pharmaceutical tablet for buccal or sublingual application comprising: about 72.8 mg epinephrine (EPBT); about 66.8 mg microcrystalline cellulose; about 7.4 mg low-substituted hydroxypropyl cellulose; and about 3 mg Magnesium stearate.

According to another embodiment of the invention, there is provided a pharmaceutical tablet for buccal or sublingual application comprising: about 24% epinephrine (EPBT); about 66% microcrystalline cellulose; about 8% low-substituted hydroxypropyl cellulose; and about 2% Magnesium stearate.

In one embodiment, there is provided herein a pharmaceutical tablet for buccal or sublingual application comprising: about 24.3% epinephrine (EPBT); about 66.4% microcrystalline cellulose; about 7.4% low-substituted hydroxypropyl cellulose; and about 2% Magnesium stearate.

In a preferred embodiment, there is provided herein a pharmaceutical tablet for buccal or sublingual application comprising: about 24.26% epinephrine (EPBT); about 66.37% microcrystalline cellulose; about 7.37% low-substituted hydroxypropyl cellulose; and about 2% Magnesium stearate. As discussed herein, this formulation is suitable children.

According to another embodiment of the invention, there is provided a pharmaceutical tablet for buccal or sublingual application comprising: about 36.4 mg epinephrine (EPBT); about 99.5 mg microcrystalline cellulose; about 11.1 mg low-substituted hydroxypropyl cellulose; and about 3 mg magnesium stearate.

According to another embodiment of the invention, there is provided a pharmaceutical tablet for buccal or sublingual application comprising: about 12% epinephrine (EPBT); about 77.5% microcrystalline cellulose; about 8.5% low-substituted hydroxypropyl cellulose; and about 2% magnesium stearate.

In a preferred embodiment, there is provided herein a pharmaceutical tablet for buccal or sublingual application comprising: about 12.1% epinephrine (EPBT); about 77.3% microcrystalline cellulose; about 8.6% low-substituted hydroxypropyl cellulose; and about 2% Magnesium stearate.

In a preferred embodiment, there is provided herein a pharmaceutical tablet for buccal or sublingual application comprising: about 12.13% epinephrine (EPBT); about 77.28% microcrystalline cellulose; about 8.59% low-substituted hydroxypropyl cellulose; and about 2% Magnesium stearate. As discussed herein, this formulation is suitable for infants.

According to another embodiment of the invention, there is provided a pharmaceutical tablet for buccal or sublingual application comprising: about 18.2 mg epinephrine (EPBT); about 116.0 mg microcrystalline cellulose; about 12.9 mg low-substituted hydroxypropyl cellulose; and about 3 mg magnesium stearate.

In one embodiment, there is provided a pharmaceutical tablet for buccal or sublingual application having a general formula as follows: about 0.5% to about 90% epinephrine; about 7.5% to about 95% filler; and about 2.5% to about 10.5% disintegrant.

In another embodiment, there is provided a pharmaceutical tablet for buccal or sublingual application having a general formula as follows: about 65% to about 75% epinephrine; about 20% to about 30% filler; and about 2.5% to about 5% disintegrant.

In yet another embodiment, there is provided a pharmaceutical tablet for buccal or sublingual application having a general formula as follows: about 43.5% to about 53.5% epinephrine; about 39.5% to about 49.5% filler; and about 2.6% to about 7.0% disintegrant.

In still another embodiment, there is provided a pharmaceutical tablet for buccal or sublingual application having a general formula as follows: about 19.3% to about 29.3% epinephrine; about 61.5% to about 71.4% filler; and about 6.8% to about 9.2% disintegrant.

In yet still another embodiment, there is provided a pharmaceutical tablet for buccal or sublingual application having a general formula as follows: about 7.1% to about 17.1% epinephrine; about 72.4% to about 82.3% filler; and about 7.9% to about 10.5% disintegrant.

In other embodiments, there is provided a pharmaceutical tablet for buccal or sublingual application in adults comprising about 40% to about 70% epinephrine (about 35 mg to about 60 mg), or comprising about 40% to about 55% epinephrine (about 35 mg to about 45 mg) or comprising about 65% to 90% epinephrine (about 55 mg to about 75 mg).

As discussed below, in certain embodiments, the pharmaceutical tablets for buccal or sublingual administration described herein can comprise an epinephrine formulation which includes excipients consisting essentially of a filler (e.g., microcrystalline cellulose (MCC)) and a disintegrant (e.g., low-substituted hydroxypropyl cellulose (L-HPC)). In certain embodiments, the filler to disintegrant ratio (i.e., filler: disintegrant) can sum to 10. As such, ratios such as 9:1, 9.5:0.5, 8:2, 7:3, and 6:4 are suitable for use in the present invention. Thus, in certain embodiments, the use of such ratios can provide rapid and complete, or substantially complete, disintegration of the buccal or sublingual tablet and can be adjusted to control the disintegration rate of the tablet. For example, the higher the disintegrant ratio, the slower the disintegration of the tablet due to lower water penetration of the tablet through capillary action. In other embodiments, the pharmaceutical tablets for buccal or sublingual administration described herein can comprise one or more fillers, one or more disintegrants, and optionally other non-essential or less essential components or excipients known in the art, for example, but by no means limited to, diluents, binders, glidants, lubricants, colorants, flavorants, coating materials and the like, as well known to one of skill in the art may be added.

In the above formulas, MCC may be added at about 20% to about 30% (about 30 mg to about 45 mg), at about 40% to about 50% (about 60 mg to about 70 mg), at about 30% to about 40% (about 45 mg to about 60 mg), at about 55% to about 65% (about 80 mg to about 100 mg), at about 65% to about 75% (about 100 mg to about 120 mg), at about 75% to about 85% (about 80 mg to about 125 mg), at about 85% to about 95% (about 125 mg to about 145 mg) or at about 10% to 95% (about 15 mg to about 145 mg).

In certain embodiments described herein, the MCC can have a particles size of less than about 700 μm. In certain other embodiments, the MCC can have a particles size of less than about 500 μm. In other embodiments, the MCC can have a particles size of ranging from about 5 μm to about 500 μm. In still other embodiments, the MCC can have a particles size of ranging from about 20 μm to about 400 μm. In yet still other embodiments, the MCC can have a particles size of ranging from about 50 μm to about 300 μm. In still yet other embodiments, the MCC can have a particles size of ranging from about 100 μm to about 200 μm. In one embodiment, the MCC can have a particles size of about 50 μm.

In some embodiments of the present invention, the MCC is Ceolus®-PH-301 (50 μm). It is of note that in alternative embodiments, other Ceolus®-PH formulations ranging in particle size from 5 μm to 500 μm may be substituted for the MCC. In yet other embodiments, other suitable MCC brands such as for example but by no means limited to: Avicel®, Elcelma®, EMCOCEL®, VIVAPUR®, VIVACEL®, SOLKA-FLOC®, Tabulose® may be utilized, having a particle size range from 7 to 300 μm.

In some embodiments, wherein MCC having a particle size less than 50 μm is added, powder flowability may have to be enhanced by the addition of other water insoluble excipients such as for example but by no means limited to Rxcipients® GL200 (silicon dioxide).

In alternative embodiments, other fillers could be substituted for, or used in addition to, MCC including, but not limited to, lactose, calcium carbonate, calcium bicarbonate, calcium phosphate, dibasic calcium phosphate, calcium sulfate, calcium silicate, cellulose powders, dextrose, dextrates, dextrans, starches, pregelatinized starches, sucrose, xylitol, lactitol, sorbitol, sodium bicarbonate, sodium chloride, polyethylene glycol, and the like.

In some embodiments, the L-HPC is added at about 4.5% to about 5.5% (about 7 mg to about 8 mg), at about 5.5% to about 7.5% (about 8 mg to about 12 mg), at about 7.5% to about 9.5% (about 12 mg to about 15 mg), or at about 2.5% to about 10.5% (about 3 mg to about 20 mg).

It is of note that in some embodiments, the L-HPC is L-HPC-LH11 having a particle size of about 50 μm. In other embodiments, the particle size may be from about 10 to about 100 μm.

In alternative embodiments, other disintegrants useful in the present invention include, but are not limited to, cross-linked celluloses, such as cross-linked sodium carboxymethyl cellulose (e.g., Ac-Di-Sol®), cross-linked carboxymethyl celluloses, or cross-linked croscarmelloses, cross-linked starches such as sodium starch glycolate (e.g., Explotab®), and a cross-linked polymers such as Crospovidone (e.g., Polyplasdone®) and may be substituted as well as any other suitable disintegrant known in the art.

In some embodiments, the tablets have a weight of about 140 to about 160 mg, and contain doses of about 5 to about 60 mg epinephrine. In other embodiments, depending on the dose of epinephrine used, tablets ranging in weight from about 20 mg to about 300 mg could be prepared. The ratios of excipients and disintegrants are adjusted to the percentages and weights described previously.

As will be appreciated by one of skill in the art, in some embodiments, the above-described formulae may be used for the synthesis or production of the active components of an epinephrine tablet and other, non-essential or less essential components or excipients known in the art, for example, but by no means limited to diluents, binders, glidants, lubricants, colorants, flavorants, secretagogues, coating materials and the like, as well known to one of skill in the art may be added.

Diluents increase bulk of the composition to facilitate compression of the tablet. As used herein, diluents include, but are not limited to, compounds such as lactose, starch, sorbitol, mannitol, dextrose, tricalcium phosphate, calcium phosphate; anhydrous lactose, spray-dried lactose; pregelatinized starch, compressible sugar, such as Di-Pac® (Amstar), hydroxypropylmethyl cellulose, hydroxypropylmethyl cellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; sodium chloride, and the like.

Binders, as used herein, refer to compounds which impart cohesive qualities to the tableted formulation and include, but are not limited to, compounds such as alginic acid and salts thereof; cellulose derivatives such as carboxymethyl cellulose, methylcellulose (e.g., Methocel®), hydroxypropylmethyl cellulose, hydroxyethyl cellulose, hydroxypropyl cellulose (e.g., Klucel®), ethyl cellulose (e.g., Ethocel®), and microcrystalline cellulose (e.g., Avicel®); microcrystalline dextrose; amylose; magnesium aluminum silicate; polysaccharide acids; bentonites; gelatin; polyvinylpyrrolidone/vinyl acetate copolymer; crosspovidone; povidone; starch, pregelatinized starch; tragacanth, dextrin, a sugar, such as sucrose (e.g., Dipac®), glucose, dextrose, molasses, mannitol, sorbitol, xylitol (e.g., Xylitab®), and lactose; a natural or synthetic gum such as acacia, tragacanth, ghatti gum, mucilage of isapol husks, polyvinylpyrrolidone (e.g., Polyvidone® CL, Kollidon® CL, Polyplasdone® XL-10), larch arabogalactan, Veegum®, polyethylene glycol, waxes, sodium alginate, and the like.

Lubricants and glidants are compounds that prevent, reduce or inhibit adhesion or friction of materials. Exemplary lubricants or glidants include, but are not limited to, stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®), a starch such as corn starch, silicone oil, a surfactant, and the like.

Flavoring agents and/or sweeteners useful in the epinephrine formulations described herein, include, but are not limited to, compounds such as acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, talin, sylitol, sucralose, sorbitol, Swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof.

It should be appreciated that there is considerable overlap between additives used in the solid dosage forms described herein. Thus, the above-listed additives should be taken as merely exemplary, and not limiting, of the types of additives that can be included in solid dosage forms of the present invention. The amounts of such additives can be readily determined by one skilled in the art, according to the particular properties desired.

In some embodiments, the tablets are compressed using die/punch diameters of 6/32″ to 11/32″ for sublingual administration. In other embodiments, die/punch sizes in the range of diameters of ⅛″ to ¾″ are used to compress sublingual epinephrine tablets. In certain other embodiments, die/punch sizes in the range of diameters of ¼″ to l/2″ are used to compress sublingual epinephrine tablets.

It is of note that the specific geometry may vary considerably and may be for example round or circular, or of other shapes, such as triangle, square, oblong, capsule-shaped or any other shape known in the art.

It is of note that flat punches, scored punches, concave punches may be used to compress buccal or sublingual epinephrine tablets. Alternatively, punches of any conceivable shape/design may be used to compress buccal or sublingual epinephrine tablets.

According to an embodiment of the invention, there is provided a pharmaceutical tablet for buccal or sublingual application consisting essentially of: about 48.5% epinephrine (EPBT); about 44.5% microcrystalline cellulose; about 5% low-substituted hydroxypropyl cellulose; and about 2% Magnesium stearate.

In another aspect of the invention, there is provided a pharmaceutical tablet for buccal or sublingual application consisting essentially of: about 72.8 mg epinephrine (EPBT); about 66.8 mg microcrystalline cellulose; about 7.4 mg low-substituted hydroxypropyl cellulose; and about 3 mg magnesium stearate.

According to the invention, there is provided a pharmaceutical tablet for buccal or sublingual application consisting of: about 48.5% epinephrine (EPBT); about 44.5% microcrystalline cellulose; about 5% low-substituted hydroxypropyl cellulose; and about 2% magnesium stearate.

In another aspect of the invention, there is provided a pharmaceutical tablet for buccal or sublingual application consisting of: about 72.8 mg epinephrine (EPBT); about 66.8 mg microcrystalline cellulose; about 7.4 mg low-substituted hydroxypropyl cellulose; and about 3 mg magnesium stearate.

According to another embodiment of the invention, there is provided a pharmaceutical tablet for buccal or sublingual application consisting essentially of: about 24.26% epinephrine (EPBT); about 66.37% microcrystalline cellulose; about 7.37% low-substituted hydroxypropyl cellulose; and about 2% magnesium stearate. As discussed herein, this formulation is suitable for children.

According to another embodiment of the invention, there is provided a pharmaceutical tablet for buccal or sublingual application consisting essentially of: about 36.4 mg epinephrine (EPBT); about 99.5 mg microcrystalline cellulose; about 11.1 mg low-substituted hydroxypropyl cellulose; and about 3 g magnesium stearate.

According to another embodiment of the invention, there is provided a pharmaceutical tablet for buccal or sublingual application consisting of: about 24.26% epinephrine (EPBT); about 66.37% microcrystalline cellulose; about 7.37% low-substituted hydroxypropyl cellulose; and about 2% magnesium stearate. As discussed about 24.26% epinephrine (EPBT); about 66.37% microcrystalline cellulose; about 7.37% low-substituted hydroxypropyl cellulose; and about 2% magnesium stearate herein, this formulation is suitable for children.

According to another embodiment of the invention, there is provided a pharmaceutical tablet for buccal or sublingual application consisting of: about 36.4 mg epinephrine (EPBT); about 99.5 mg microcrystalline cellulose; about 11.1 mg low-substituted hydroxypropyl cellulose; and about 3 mg magnesium stearate.

In another aspect of the invention, there is provided a method of preparing an epinephrine tablet for buccal or sublingual administration comprising: about 12.13% epinephrine (EPBT); about 77.28% microcrystalline cellulose; about 8.59% low-substituted hydroxypropyl cellulose; and about 2% magnesium stearate. As discussed about 12.13% epinephrine (EPBT); about 77.28% microcrystalline cellulose; about 8.59% low-substituted hydroxypropyl cellulose; and about 2% magnesium stearate. As discussed herein, this formulation is suitable for infants.

According to another embodiment of the invention, there is provided a pharmaceutical tablet for buccal or sublingual application consisting of: about 18.2 mg epinephrine (EPBT); about 116.0 mg microcrystalline cellulose; about 12.9 mg low-substituted hydroxypropyl cellulose; and about 3 mg magnesium stearate.

In another aspect of the invention, there is provided a method of preparing a mixture of any one of the above-described formulae; and compressing unit dosage portions of the mixture to about 24 kN, thereby producing a tablet. As discussed above, the unit dosage weight is typically 150 milligrams, although other suitable sizes may also be used and are within the scope of the invention.

ii. Methods of Use of Fast-Disintegrating Buccal or Sublingual Tablets Comprising Epinephrine

Also provided herein are methods for the treatment of a patient in need of epinephrine therapy comprising the administration a fast disintegrating buccal or sublingual tablet described herein. In certain embodiments, the methods comprise treating a patient having, or suspected of having, an allergic emergency (e.g., anaphylaxis) with a fast disintegrating buccal or sublingual tablet described herein. In other embodiments, the methods comprise treating a patient having, or suspected of having, asthma (e.g., bronchial asthma) with a fast disintegrating buccal or sublingual tablet described herein. In still other embodiments, the methods comprise treating a patient having, or suspected of having, a cardiac event (e.g., cardiac arrest, asystole, or ventricular defibrillation) with a fast disintegrating buccal or sublingual tablet described herein.

i. Age Specific Buccal or Sublingual Epinephrine Tablets and Treatment Methods Based Thereon

In certain embodiments of the present invention, fast disintegrating buccal or sublingual tablet described herein are provided which are formulated for specific administration to patients in certain pre-determined age groups, e.g., adults, adolescents, children, infants or newborns. In other embodiments, the present invention provides methods of treatment specific for certain pre-determined age groups, e.g., adults, adolescents, children, infants or newborns.

In one embodiment of the present invention, there is provided a pharmaceutical fast disintegrating tablet for buccal or sublingual application in adults comprising about 25 mg to about 75 mg epinephrine.

In another embodiment, there is provided a pharmaceutical fast disintegrating tablet for buccal or sublingual application in adolescents comprising about 25 mg to about 40 mg epinephrine (about 35% to about 45% epinephrine).

In another embodiment, there is provided a pharmaceutical fast disintegrating tablet for buccal or sublingual application in children comprising about 10 mg to about 25 mg epinephrine (about 20% to about 35% epinephrine).

In another embodiment, there is provided a pharmaceutical fast disintegrating tablet for buccal or sublingual application in infants comprising about 5 mg to about 10 mg epinephrine (about 10% to about 15% epinephrine).

In another embodiment, there is provided a pharmaceutical fast disintegrating tablet for buccal or sublingual application in newborns or small infants comprising about 0.5 mg to about 5 mg epinephrine (about 2% to about 8% epinephrine).

As will be appreciated by one of skill in the art, there are differing opinions as to the appropriate age ranges for the above-listed age groups. However, for purposes of discussion, it is assumed that Newborns refers to 0-3 months; infants, 3 months-2 years; children, 2-12 years; adolescents 12-18 years. It is important to note that EP dose is typically based on weight. As such, one advantage of the tablet form is that a wider range of doses can be provided compared to the Epi-Pen or other auto-injector forms which administer a fixed dosage. For example, the tablets could be scored so that the tablet can be broken into pre-selected sizes, thereby allowing greater flexibility in the dosage administered.

In one embodiment, the present invention provides a method for the treatment of anaphylaxis in an adult comprising step of administering a dose of a buccal or sublingual fast disintegrating tablet comprising about 25 mg to about 75 mg epinephrine.

In another embodiment, the present invention provides a method for the treatment of anaphylaxis in an adolescent comprising step of administering a dose of a buccal or sublingual fast disintegrating tablet comprising about 25 mg to about 40 mg epinephrine.

In yet another embodiment, the present invention provides a method for the treatment of anaphylaxis in an child comprising step of administering a dose of a buccal or sublingual fast disintegrating tablet comprising about 10 mg to about 25 mg epinephrine.

In still another embodiment, the present invention provides a method for the treatment of anaphylaxis in an infant comprising step of administering a dose of a buccal or sublingual fast disintegrating tablet comprising about 5 mg to about 10 mg epinephrine.

In still yet another embodiment, the present invention provides a method for the treatment of anaphylaxis in a newborn comprising step of administering a dose of a buccal or sublingual fast disintegrating tablet comprising about 0.5 mg to about 5 mg epinephrine.

iii. Fast-Disintegrating Sublingual Tablets Comprising Epinephrine Show Long Term Stability

In certain embodiments, the sublingual epinephrine tablets described herein are stable for at least 2 years. In certain embodiments, the sublingual epinephrine tablets can have at least 90 percent of the initial epinephrine content after 24 months at 25° C. In other embodiments, the sublingual epinephrine tablets can have at least 95 percent of the initial epinephrine content after 24 months at 25° C. In still other embodiments, the sublingual epinephrine tablets can have at least 97.5 percent of the initial epinephrine content after 24 months at 25° C. In one embodiment, the sublingual epinephrine tablet comprises 10 mg EP. In another embodiment, the sublingual epinephrine tablet comprises 20 mg EP. In yet another embodiment, sublingual epinephrine tablet comprises 40 mg EP.

In certain other embodiments, the sublingual epinephrine tablets can have at least 90 percent of the initial epinephrine content after 20 months at 25° C. In other embodiments, the sublingual epinephrine tablets can have at least 95 percent of the initial epinephrine content after 20 months at 25° C. In still other embodiments, the sublingual epinephrine tablets can have at least 97.5 percent of the initial epinephrine content after 20 months at 25° C. In one embodiment, the sublingual epinephrine tablet comprises 10 mg EP. In another embodiment, the sublingual epinephrine tablet comprises 20 mg EP. In yet another embodiment, sublingual epinephrine tablet comprises 40 mg EP.

In still other embodiments, the sublingual epinephrine tablets can have at least 90 percent of the initial epinephrine content after 12 months at 25° C. In other embodiments, the sublingual epinephrine tablets can have at least 95 percent of the initial epinephrine content after 12 months at 25° C. In still other embodiments, the sublingual epinephrine tablets can have at least 97.5 percent of the initial epinephrine content after 12 months at 25° C. In one embodiment, the sublingual epinephrine tablet comprises 10 mg EP. In another embodiment, the sublingual epinephrine tablet comprises 20 mg EP. In yet another embodiment, sublingual epinephrine tablet comprises 40 mg EP.

In yet other embodiments, the sublingual epinephrine tablets can have at least 90 percent of the initial epinephrine content after 6 months at 25° C. In other embodiments, the sublingual epinephrine tablets can have at least 95 percent of the initial epinephrine content after 6 months at 25° C. In still other embodiments, the sublingual epinephrine tablets can have at least 97.5 percent of the initial epinephrine content after 6 months at 25° C. In one embodiment, the sublingual epinephrine tablet comprises 10 mg EP. In another embodiment, the sublingual epinephrine tablet comprises 20 mg EP. In yet another embodiment, sublingual epinephrine tablet comprises 40 mg EP.

In certain embodiments, the sublingual epinephrine tablets can have at least 90 percent of the initial epinephrine content after 24 months at 5° C. In other embodiments, the sublingual epinephrine tablets can have at least 95 percent of the initial epinephrine content after 24 months at 5° C. In still other embodiments, the sublingual epinephrine tablets can have at least 97.5 percent of the initial epinephrine content after 24 months at 5° C. In one embodiment, the sublingual epinephrine tablet comprises 10 mg EP. In another embodiment, the sublingual epinephrine tablet comprises 20 mg EP. In yet another embodiment, sublingual epinephrine tablet comprises 40 mg EP.

In certain other embodiments, the sublingual epinephrine tablets can have at least 90 percent of the initial epinephrine content after 20 months at 5° C. In other embodiments, the sublingual epinephrine tablets can have at least 95 percent of the initial epinephrine content after 20 months at 5° C. In still other embodiments, the sublingual epinephrine tablets can have at least 97.5 percent of the initial epinephrine content after 20 months at 5° C. In one embodiment, the sublingual epinephrine tablet comprises 10 mg EP. In another embodiment, the sublingual epinephrine tablet comprises 20 mg EP. In yet another embodiment, sublingual epinephrine tablet comprises 40 mg EP.

In still other embodiments, the sublingual epinephrine tablets can have at least 90 percent of the initial epinephrine content after 12 months at 5° C. In other embodiments, the sublingual epinephrine tablets can have at least 95 percent of the initial epinephrine content after 12 months at 5° C. In still other embodiments, the sublingual epinephrine tablets can have at least 97.5 percent of the initial epinephrine content after 12 months at 5° C. In one embodiment, the sublingual epinephrine tablet comprises 10 mg EP. In another embodiment, the sublingual epinephrine tablet comprises 20 mg EP. In yet another embodiment, sublingual epinephrine tablet comprises 40 mg EP.

In yet other embodiments, the sublingual epinephrine tablets can have at least 90 percent of the initial epinephrine content after 6 months at 5° C. In other embodiments, the sublingual epinephrine tablets can have at least 95 percent of the initial epinephrine content after 6 months at 5° C. In still other embodiments, the sublingual epinephrine tablets can have at least 97.5 percent of the initial epinephrine content after 6 months at 5° C. In one embodiment, the sublingual epinephrine tablet comprises 10 mg EP. In another embodiment, the sublingual epinephrine tablet comprises 20 mg EP. In yet another embodiment, sublingual epinephrine tablet comprises 40 mg EP.

In certain embodiments, the sublingual epinephrine tablets can have at least 90 percent of the initial epinephrine content after 24 months at 5° C. with nitrogen flushing. In other embodiments, the sublingual epinephrine tablets can have at least 95 percent of the initial epinephrine content after 24 months at 5° C. with nitrogen flushing. In still other embodiments, the sublingual epinephrine tablets can have at least 97.5 percent of the initial epinephrine content after 24 months at 5° C. with nitrogen flushing. In one embodiment, the sublingual epinephrine tablet comprises 10 mg EP. In another embodiment, the sublingual epinephrine tablet comprises 20 mg EP. In yet another embodiment, sublingual epinephrine tablet comprises 40 mg EP.

In certain other embodiments, the sublingual epinephrine tablets can have at least 90 percent of the initial epinephrine content after 20 months at 5° C. with nitrogen flushing. In other embodiments, the sublingual epinephrine tablets can have at least 95 percent of the initial epinephrine content after 20 months at 5° C. with nitrogen flushing. In still other embodiments, the sublingual epinephrine tablets can have at least 97.5 percent of the initial epinephrine content after 20 months at 5° C. with nitrogen flushing. In one embodiment, the sublingual epinephrine tablet comprises 10 mg EP. In another embodiment, the sublingual epinephrine tablet comprises 20 mg EP. In yet another embodiment, sublingual epinephrine tablet comprises 40 mg EP.

In still other embodiments, the sublingual epinephrine tablets can have at least 90 percent of the initial epinephrine content after 12 months at 5° C. with nitrogen flushing. In other embodiments, the sublingual epinephrine tablets can have at least 95 percent of the initial epinephrine content after 12 months at 5° C. with nitrogen flushing. In still other embodiments, the sublingual epinephrine tablets can have at least 97.5 percent of the initial epinephrine content after 12 months at 5° C. with nitrogen flushing. In one embodiment, the sublingual epinephrine tablet comprises 10 mg EP. In another embodiment, the sublingual epinephrine tablet comprises 20 mg EP. In yet another embodiment, sublingual epinephrine tablet comprises 40 mg EP.

In yet other embodiments, the sublingual epinephrine tablets can have at least 90 percent of the initial epinephrine content after 6 months at 5° C. with nitrogen flushing. In other embodiments, the sublingual epinephrine tablets can have at least 95 percent of the initial epinephrine content after 6 months at 5° C. with nitrogen flushing. In still other embodiments, the sublingual epinephrine tablets can have at least 97.5 percent of the initial epinephrine content after 6 months at 5° C. with nitrogen flushing. In one embodiment, the sublingual epinephrine tablet comprises 10 mg EP. In another embodiment, the sublingual epinephrine tablet comprises 20 mg EP. In yet another embodiment, sublingual epinephrine tablet comprises 40 mg EP.

In other embodiments, the sublingual epinephrine tablets display not more than a 5 percent decrease in epinephrine content after up to 24 months at 25° C. In still other embodiments, the sublingual epinephrine tablets display not more than a 5 percent decrease in epinephrine content after up to 24 months at 5° C. In yet other embodiments, the sublingual epinephrine tablets display not more than a 5 percent decrease in epinephrine content after up to 24 months at 5° C. with nitrogen flushing. In one embodiment, the sublingual epinephrine tablet comprises 10 mg EP. In another embodiment, the sublingual epinephrine tablet comprises 20 mg EP. In yet another embodiment, sublingual epinephrine tablet comprises 40 mg EP.

iv. In Vivo Activity of Fast-Disintegrating Sublingual Tablets Comprising Epinephrine

We have demonstrated herein that the administration of sublingual epinephrine tablets to achieve therapeutic levels of epinephrine in vivo is feasible. In addition, we have shown that the sublingual tablet formulations described herein, when used in a rabbit model, can achieve epinephrine plasma concentrations which are not significantly different from the 0.3 mg epinephrine IM dose in the thigh muscle, the currently recommended emergency treatment for anaphylaxis

In certain aspects of the present invention, fast disintegrating buccal or sublingual epinephrine tablets can be developed to deliver various doses of epinephrine by the buccal or sublingual route of administration.

In other aspects of the present invention, the epinephrine dose administered sublingually in Formulation I-D in a rabbit model will achieve plasma epinephrine concentrations similar to those obtained following the intramuscular injection of 0.3 mg of epinephrine via EpiPen® in the thigh muscle. A 0.3 mg dose of epinephrine administered intramuscularly in the thigh muscle is the currently recommended treatment for anaphylaxis in adult patients. The dose of epinephrine for the emergency treatment of anaphylaxis is 0.01 mg/kg up to a maximum of 0.3 mg in patients greater than 30 kg. In Europe, doses as high as 0.5 mg are recommended. As described above, the “correct” plasma epinephrine concentrations for emergency treatment of anaphylaxis have never been determined. However, the 0.3 to 0.5 mg doses have become accepted and mandated based on 60 to 70 years experience and anecdotal evidence. Patients die even when epinephrine is administered, possibly by the SC route which we have demonstrated produces lower plasma epinephrine concentrations than the IM route, or the epinephrine injection was administered too late following the onset of the anaphylactic episode. Also, patients experiencing anaphylaxis may not die, and may fully recover with no adverse effects even if epinephrine was NOT administered.

Thus, in certain embodiments, formulation I-D provides an alternative, non invasive method for the treatment of anaphylaxis in adult patients. In other embodiments, smaller doses, e.g., formulations I-B or I-C may be used for pediatric patients, as discussed above.

These fast disintegrating sublingual epinephrine tablets provide a safe, user friendly, and effective alternative route of administration of epinephrine for the emergency treatment of anaphylaxis away from a health care facility. These sublingual epinephrine tablets provide the advantage of a wider range of dosage strengths for improved safety in infants and children, whereas only EpiPen, 0.3 mg and EpiPen Jr 0.15 mg and Twinject 0.3 mg and 0.15 mg are currently available in autoinjectors. In addition, the sublingual epinephrine tablets readily provide the opportunity for multiple doses, as is often required for the treatment of anaphylaxis, especially when the incident occurs in a remote area, far from a health care facility.

### EXAMPLES

The invention will now be further explained by way of examples. However, the invention is not necessarily limited by the examples.

### (a) Example I

### Materials

(−)-Epinephrine (+) bitartrate (EPBT) was purchased from Sigma-Aldrich (St. Louis, Mo., USA). The following excipients were used: Ceolus® PH-301 (microcrystalline cellulose, MCC) with a mean particle size of 50 μm (Asahi Kasei Chemicals Corp, Tokyo, Japan) and low-substituted hydroxypropyl cellulose (L-HPC-LH11) with a mean particle size of 50 μm (Shin-Etsu Chemical Co, Tokyo. Japan). The magnesium stearate (MS) was purchased from Mallinckrodt Baker (Phillipsburg, N.J., USA). As will be apparent to one of skill in the art, particle size of magnesium state does not seem to be critical but it is usually purchased as a very fine powder because it is used as a lubricant and must be distributed thoroughly and uniformly in order to result in a uniform flow of powder during tablet formation to result in tablets of uniform weight and epinephrine content.

### (b) Example 2

### Preparation of Tablets

Four tablet formulations A, B, C, and D containing 0%, 6%, 12% and 24% of EPBT, respectively, equivalent to 0, 5, 10, and 20 mg of EP respectively, were prepared by direct compression (Table I). The total weight of the compressed EPBT tablets was maintained at 150 mg. Formulations A, B, C, and D were prepared by mixing the proposed EPBT amount with the total quantity of MCC and two-thirds of the quantity of L-HPC by using a three dimensional manual mixer (Inversina®, Bioengineering AG, Switzerland) for 4.5 minutes. The MCC:L-HPC ratio in each of the final tablet formulations was always maintained at 9:1 (Ishikawa et al., 2001; Watanabe et al., 1995; Bi et al., 1996; Bi et al., 1999). It is of note that the total should always be 10, i.e. 9:1, 8:2, 7:3. All of the magnesium stearate (MS) and the remaining one-third of the quantity of L-HPC were added 30 seconds before the end of mixing.

Each tablet formulation was compressed at a range of forces (CF) as shown in Table II. An 11/32 inch die with a flat, scored face, bevel edge upper punch and a bevel edge lower punch were selected based on results from our previous study (Rawas-Qalaji et al., 2005, American Association of Pharmaceutical Scientists Journal 7(52): Abstract W5220). The flat-scored tablets were compressed using a Manesty®—F3 single-punch tablet press machine (Liverpool, UK).

### (c) Example 3

### Evaluation of Tablet Characteristics

Each batch of tablets was collected into a stainless steel beaker. Tablet weight variation and drug content uniformity was measured using USP methods and criteria (USP/NF, 2003, Physical Test: Uniformity of Dosage Units, United States Pharmacopeial Convention, Inc: Rockville, Md.). Drug content was analyzed using HPLC-UV (Waters Corp., Milford, Mass.) and tablet friability was measured using a USP friability instrument (Parma Test Apparatebau QmbH, Heinburg, Germany). Six tablets were selected randomly from each formulation batch and tested for tablet hardness, disintegration time, and wetting time. The mean±standard deviation (SD), and coefficient of variation percentage (CV %) were calculated.

Hardness (H): The H or the crushing tolerance of tablets, the force that applied on the tablet diameter to break them, was measured by an Erweka® hardness tester (Heusenstanun, Germany). As discussed above, if the tablet either contains the improper excipients, or excipients in the incorrect proportions, or if it is compressed by too much force, then it will not disintegrate rapidly. If the tablet is formulated with the correct excipients in the correct proportions but compressed with insufficient force, then the tablets readily disintegrate into smaller pieces or even into powder with routine shipping or handling, and would be useless for patient administration.

Disintegration Time (DT): The DT was measured using a stopwatch to record the time required for the tablet to disintegrate completely into fine particles in 2 ml of distilled water in a 10 ml glass test tube, with no agitation. As will be appreciated by one of skill in the art, if the DT was too long, a patient experiencing anaphylaxis may not be able to retain a tablet sublingually for many minutes, so it would be very important that the tablet disintegrate as quickly as possible and release the epinephrine so it could be absorbed sublingually as quickly as possible.

Wetting Time (WT): Tablet WT was measured by a procedure similar to that reported by Bi et al. (Bi et al., 1996). The tablet was placed at the center of 2 layers of absorbant paper fitted in a rectangular plastic dish (11 cm×7.5 cm). After the paper was thoroughly wetted with distilled water, excess water was completely drained out of the dish. The time required for the water to diffuse from the wetted absorbant paper throughout the entire tablet was then recorded by using a stopwatch. As will be appreciated by one of skill in the art, the WT cannot be too long, as it is very important that the tablet disintegrate as quickly as possible and release the epinephrine so it could be absorbed sublingually as quickly as possible.

Data Analysis and Curve Fitting: All results were reported as mean±standard deviation (SD) (n=5) and analyzed by plotting H, DT, and WT versus CF; DT and WT versus H and WT versus DT. The relationships were fitted to appropriate equations using Axum 5.0C (MathSof, Inc.) and NCSS(NCSS, Kaysville, Utah) softwares. The parameters of each equation and the correlation of fit (R2) were calculated using NCSS and Excel 2000 (Microsoft Corporation) softwares.

The powders from all four (A, B, C, D as set forth in Table I) as well as other three (II-E, III-F, IV-G) formulations resulted in good mixing, flowability, and compressibility characteristics. Tablets manufactured from each formulation were within USP specifications for weight variation and drug content uniformity (USP/NF, 2003).

(i) Hardness:

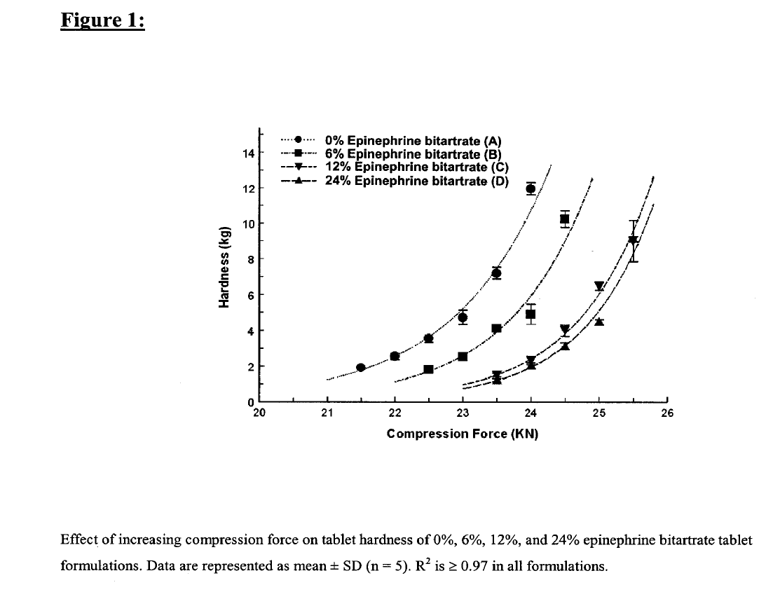

The hardness (H) characteristics for each formulation achieved for a series of increasing CF values are shown in Table II. The effect of increasing the CF on the tablet H for each formulation is demonstrated in FIG. 1. A linear increase in the CF resulted in an exponential increase in the tablet H in the four different formulations. The increase in CF possibly reduced the tablet porosity due to a closer rearrangement and compaction of the particles resulting in a harder tablet (Bi et al., 1996; Bi et al., 1999; Marshall, 1986 in The Theory and Practice of Industrial Pharmacy (Lachman et al. eds), Lea & Febiger: Philadelphia). The increase in the tablet H vs. increasing CF can be described by Equation I, where X is CF and Y is H. The equation parameters a and b for the four formulations are shown in Table III.
Y=ae^{bX} (I)

As EPBT load increased, higher CF were required to achieve a range of H comparable to A formulation (0% EPBT). This may be due to the poor compressibility of EPBT, which can interfere with, and reduce the formation of, hydrogen bonds between MCC particles (Bi et al., 1996). The higher the EPBT drug load, the greater the interference with the interparticle hydrogen bonds formation and the higher compression force required to increase the contact points between powder particles in order to maintain the desired range of tablet hardness. Similar results have been reported by Watanbe et al (Watanabe et al., 1995), Bi et al (Bi et al., 1996; Bi et al., 1999), Ishikawa et al (Ishikawa et al., 2001), Sugimoto et al (Sugimoto et al., 2001, Pharm Dev Technol 6: 487-493), and Schiermeier et al (Scheirmeier and Schmidt, 2002, Eur J Pharm Sci 15: 295-305) for other medications.

### (d) Example 4

### Disintegration and Wetting Time

In the USP disintegration test for sublingual tablets, the disintegration apparatus for oral tablets is used without the covering plastic disks (USP/NF, 1990, Physical Tests: Disintegration, United States Pharmacopeial Convention: Rockville, Md.) and 2 minutes is specified as the acceptable time limit for tablet disintegration (USP/NF, 1990, Official Monographs: Nitroglycerin Tablets, United States Pharmacopeial Convention: Rockville, Md.). The design of the apparatus, the disintegration time, and the evaluation procedure specified in the USP for the disintegration of sublingual tablets were not suitable for these fast-disintegrating or fast-dissolving tablets which disintegrate so rapidly that differences in the disintegration time cannot be measured accurately using the standard USP apparatus.

Another apparatus to detect the differences in tablet disintegration time was designed by Bi et al (Bi et al., 1996). The speed of the apparatus paddle is 100 rpm and the volume of the immersion fluid is 900 ml. These conditions do not reflect the in vivo sublingual conditions where a very limited volume (0.35-1.0 nl/min) of saliva is available under normal conditions with a maximum of 5-7 ml/min after stimulation (Diem and Lentner, 1971, Scientific Tables, Ciba-Geigy Limited: Basle, Switzerland). Also, the agitation in the immersion fluid created by the paddle rotation, which would be absent in the sublingual cavity, can enhance tablet disintegration and reduce the actual tablet disintegration time compared to what might be expected in the sublingual cavity.

More complicated procedures have been used to predict the disintegration time of fast disintegrating or dissolving tablets by using a texture analyzer (Abdelbary et al., 2005, Int J Pharm 292: 29-41; el-Arini and Clas, 2002, Pharm Dev Technol 7: 361-371; Dor and Fix, 2000, Pharm Dev Technol 5: 575-577).

We developed a relatively simple method with rigorous requirements to evaluate the DT of rapidly disintegrating tablets. The tablet was dropped into a 10 ml glass test tube (1.5 cm diameter), which contained 2 ml distilled water, and the time required for complete tablet disintegration into fine particles was observed visually and recorded using a stopwatch. The visual inspection was enhanced by gently rotating the test tube at a 45° angle without agitation to distribute any tablet particles that might mask any remaining undisintegrated portion of the tablets, and potentially interfere with visual inspection.

The diameter of the test tube is smaller than the diameter of sublingual area in humans (3-4 cm). The larger sublingual area in humans might actually enhance rather than reduce tablet disintegration. The 1.5 cm diameter of the 10 ml test tube does compare to the sublingual cavity in small laboratory animals such as rabbits, which can be used in in vivo studies (Gu et al., 2002). The small volume of water used for tablet disintegration evaluation approximates the volume of saliva secreted under normal conditions. The relatively small sublingual area, the small volume of saliva available in the mouth, and the non-agitated environment under the human tongue are simulated by this in vitro disintegration test.

The wetting test designed by Bi et al (Bi et al., 1996) compares with the conditions in the sublingual area of humans and animals. We therefore used this test, with modifications in the size and the type of dish used, and in the volume of water used, as previously described.

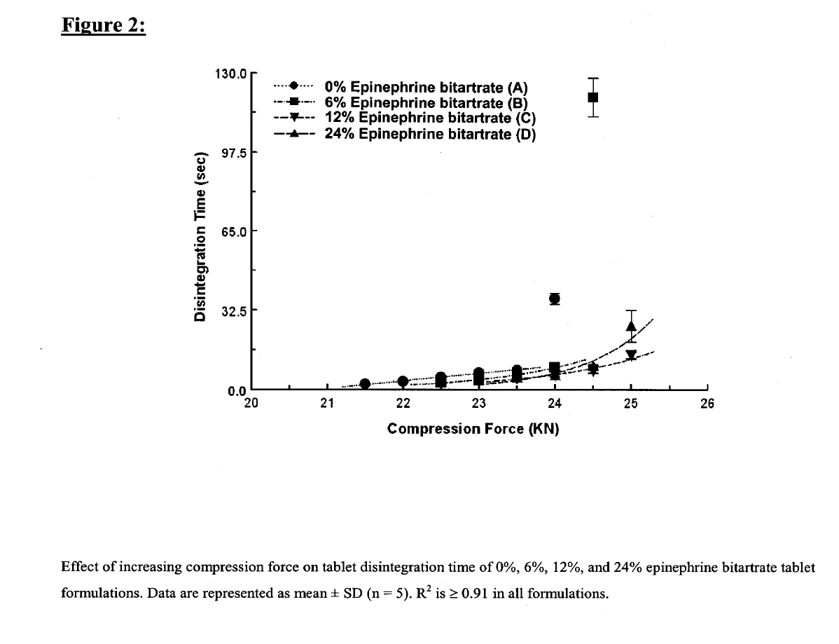

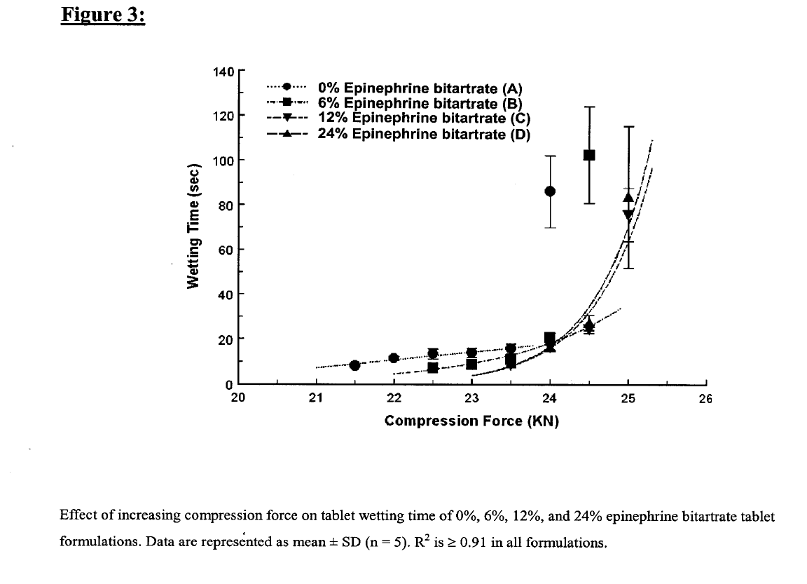

The results of the disintegration and wetting tests for each formulation following a range of increasing CF values are shown in Tables IV and V, respectively. Formulation A demonstrated an initial linear increase in the DT and WT (FIGS. 2 and 3), despite the exponential increase in the tablet hardness following the linear increase in the CF. When CF was greater than 23.5 kN, dramatic non-linear increases in DT and WT occurred. Below CF 23.5 kN the linear increase in the tablet DT and WT can be described by Equation II, where X is CF and Y is DT or WT. The equation parameters a and b for the four formulations are shown in Table III.
Y=bX−a (II)

When EPBT load was increased for formulations B, C, and D, an exponential increase in the DT and WT occurred following a linear increase in the CF up to 24 kN for B and 25 kN for C and D (FIGS. 2 and 3). The DT increased dramatically and non-exponentially when CF was greater than 24 kN for formulation D. Formulations C and D showed incomplete disintegration and wetting when CF was greater than 25 kN. The exponential increase in the tablet DT and WT can be described by Equation I, where X is CF and Y is DT or WT. The equation parameters a and b for the four formulations are shown in Table III.

Increasing CF will result in increased particle contact and reduced tablet porosity. The degree of tablet porosity plays an important role in tablet wetting and disintegration. The pores in the tablet form capillary pathways throughout the tablet that allows water penetration for complete and fast wetting of the tablet (Watanabe et al. 1995; Bi et al., 1996; Hedenus et al., 2000, Int J Pharm 202: 141-149). When water reaches the uniformly distributed super-disintegrant throughout the tablet, the super-disintegrant expands and swells to cause tablet rupture and complete tablet disintegration into smaller particles. This relationship between compression force and tablet porosity and its effect on tablet disintegration and wetting have been previously described (Watanabe et al., 1995; Bi et al., 1996; Bi et al., 1999; Sugimoto et al., 2001; Schiermeier et al., 2002).

Another important factor that can affect tablet disintegration and wetting is the degree of bond deformation upon compaction. The MCC exhibits both elastic and plastic deformation (Marshall, 1986). Upon increasing the CF, the main type of deformation initially would be an elastic deformation, where particles rearrange to form a compact. Once the compression force exceeds the elastic deformation forces, plastic deformation would be the main type of deformation causing closer and irreversible particle rearrangement. When exposed to small amounts of water, tablets that exhibited elastic deformation will demonstrate a fast disintegration and wetting times because the massive expansion of the super-disintegrant will be able to break the bonds formed during compression. On the other hand, tablets that exhibited plastic deformation will demonstrate a slower DT and WT or will not disintegrate at all. This occurs because of the closer particle arrangement that results in the formation of numerous, stronger interparticle bonds. In addition, the low tablet porosity limits water penetration and makes the role of super-disintegant more difficult or even impossible under high compression forces.

This theory could explain why tablets from all formulations demonstrated initial rapid DT and WT (FIGS. 2 and 3), despite the initial exponential increase in H as the CF increased linearly (FIG. 1). This may be due to elastic deformation. The dramatic increase in the DT and WT (FIGS. 2 and 3) that result due to the exponential increase in the H (FIG. 1) as the CF exceeded certain critical values probably represents plastic deformation.

The range of tablet H (Table II) of formulations C (mean±SD, 2.3±0.2-6.5±0.2) and D (2.0±0.2-4.5±0.1) that results in complete tablet disintegration and wetting was smaller than for formulations A and B. Increasing the EPBT load increased DT and WT dramatically at higher compression forces resulting in increasing tablet hardness, possibly due to the reduction in the capillary action as a result of low porosity of the compacted EPBT and the higher CF required to form a hard tablet compact.

(i) Relationship Between Hardness and Disintegration/Wetting Time:

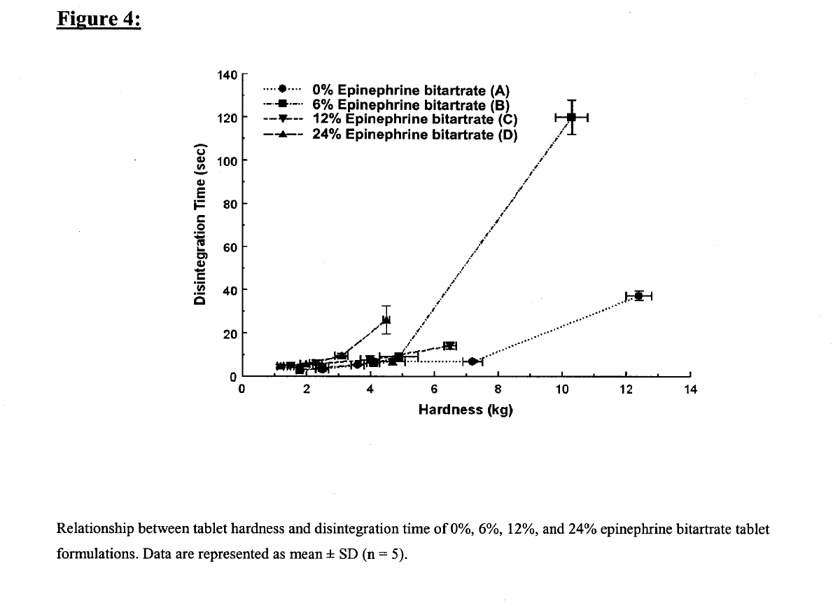

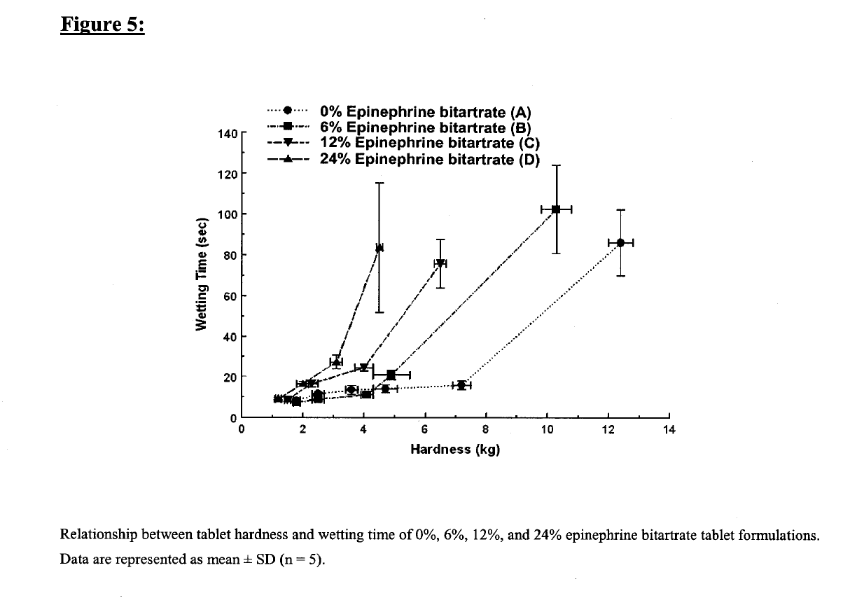

The relationship between tablet H and the resulting DT and WT of each formulation are shown in FIGS. 4 and 5.

The DT of formulation A was maintained <10 sec (6.8±0.4 sec) when the tablet H was ≦7.2±0.3 Kgf (FIG. 4), despite the exponential increase in tablet hardness. This small increase in DT as the tablet H was increased in formulation A makes it an ideal candidate to be loaded with increasing doses of EPBT.

Loading formulation A with increasing EPBT load as designated by formulations B, C, and D did not affect DT significantly at low tablet hardness (FIG. 4). The DT was maintained below 10 sec at tablet hardness ≦4.9±0.6, ≦4.0±0.3, and ≦3.1±0.2 Kgf for formulations B, C, and D, respectively (Tables II and IV). Further increases in the tablet hardness up to 6.5±0.2 and 4.5±0.1 Kgf for formulations C and D, respectively, still resulted in a rapid DT (14.0±1.4 and 26.0±6.4 sec, respectively). Formulations B, C, and D achieved rapid tablet disintegration times (FIGS. 2 and 3) without compromising tablet hardness. Tablet hardness of ≧3 Kgf has been reported to withstand shipping and handling (Fell and Newton, 1970, J Pharm Sci 59: 688-691).

Similar results were obtained upon plotting tablet H against WT for each formulation. The WT of formulation A was maintained <30 sec, despite the exponential increase in the tablet hardness up to 7.2±0.3 Kgf (FIG. 5). In contrast with increasing EPBT loads for the other formulations, a rapid WT (<30 sec) required that tablet hardness be maintained at ≦4.9±0.6, ≦4.0±0.3, and ≦3.1±0.2 Kgf for formulations B, C, and D, respectively (Tables II and V).

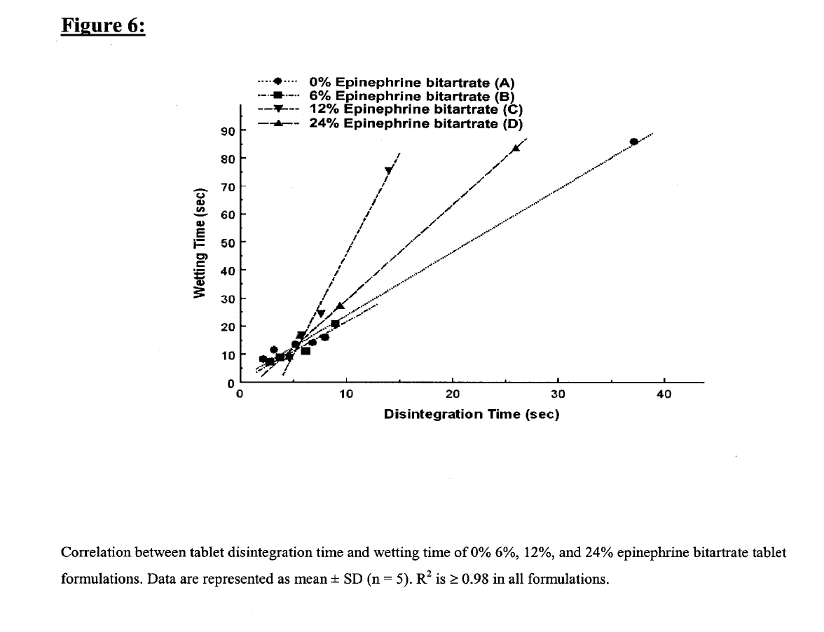

The agreement between the DT and WT of different formulations is due to the linear correlation between DT and WT (FIG. 6), where the degree of tablet porosity appears to be the common factor. Equation II demonstrates this correlation (where X is DT and Y is WT). The equation parameters (a and b) of the five formulations are shown in Table III.
Y=bX−a (II)

The linear correlation between DT and WT was also reported by Bi et al (Bi et al., 1996) and Aly et al (Aly et al., 2005, Pharmaceutical Technology 68-78).

In another example, epinephrine sublingual bioavailability from four formulations containing various excipients which have similar in vitro tablet characteristics were evaluated in comparison to EP 0.3 mg IM in the thigh from EpiPen using a validated rabbit model (Gu et al., 1999, Biopharm Drug Dispos 20: 401-405; Gu et al., 2002, Biopharm Drug Dispos 23: 213-216; Simons et al., 2000, J Allergy Clin Immunol 105: 1025-1030).

### (e) Example 5

### Materials

(−)-Epinephrine (+) bitartate (EPBT), (−)-3,4-dihydroxy-α-[(methylamino)methyl]benzyl alcohol (+)-tartrate (1:1) salt, was purchased from Sigma-Aldrich (St. Louis, Mo.). The following excipients were used: Ceolus® (microcrystalline cellulose, MCC) PH-301, PH-M-06 and KG-802 (Asahi Kasei Chemicals Corp, Tokyo, Japan), RxCipient® FM1000 (calcium silicate) (Huber Engineered Materials, Havre de Grace, Md.), and Pearlitol® 400 DC, mannitol, (Roquette America, Inc., Keokuk, Iowa), as fillers; substituted hydroxypropyl cellulose (L-HPC-LH11) (Shin-Etsu Chemical Co, Tokyo, Japan) and Polyplasdone® XL-10, crospovidone, (ISP Technologies, INC., Wayne, N.J.), as superdisintegrants; Pharmaburst®, a patent formula, (SPI Pharma, New Castle Del.), a ready to use formula for fast-disintegrating tablets; RxCipient® GL200, silicon dioxide, (Huber Engineered Materials, Havre de Grace, Md.), as a glidant; magnesium stearate (MS) was purchased from Mallinckrodt Baker (Phillipsburg, N.J.) and PRUV®, sodium stearyl fumerate (SSF), (JRS Pharma LP, Patterson, N.J.), as lubricants.

### (f) Example 6

### Preparation of Tablets

Four tablet formulations, I-D, II-E, III-F and IV-G containing 48.51% of EPBT, equivalent to 40 mg of EP, were prepared by direct compression (Table XII). The total weight of the compressed tablets was maintained at 150 mg. These tablets were prepared by mixing the pre-calculated excipient quantities using a three dimensional manual mixer (Inversina®), Bioengineering AG, Switzerland). The MCC:L-HPC in I-D and II-E formulations was always maintained at 9:1 as discussed above. All of the MS and SSF were added at the end of mixing.

Each tablet formulation was compressed using an 11/32″ die, a flat scored face, bevel edge upper punch, and a flat, bevel edge lower punch, although, as discussed above, any suitable sized and shaped may be utilized. The tablets were prepared at a compression force (CF) as discussed above and using a Manesty®X-F3 single-punch tablet press machine (Liverpool, UK).

In Vitro Evaluation of Tablet Characteristics

Each batch of ˜200 tablets was collected into a stainless steel beaker. Tablet weight variation, drug content uniformity, and tablet friability was measured using USP methods and criteria, as discussed above. Six tablets were selected randomly from each formulation batch and tested for tablet hardness, disintegration time, and wetting time. The mean, standard error (SEM), and coefficient of variation percentage (CV %) were calculated.

Hardness, Disintegration time and wetting time were determined as discussed above.

### (g) Example 7

### Effect of Water-Soluble Excipients on EP Solubility

The dissolution of 7.3 mg of EPBT in 100 μl of water and 100 μl of a saturated solution of mannitol, equivalent to 40 mg EP in 1 ml of saliva was monitored over 5 minutes using a microscope (10× power) (Nikon YS 100, Nikon Canada Inc., ON, Canada) equipped with a digital camera (Sony 3-CCD, DXC-390P, Sony Electronics Inc., NJ) using Northern Eclipse V6.0 software (Empix Imaging, Inc., ON, Canada). The 1 ml saliva was calculated based on the normal salivary secretion in humans, 0.2 ml/min, for 5 minutes, as discussed above.

### In Vivo Methods

In a prospective, controlled, 5-way crossover study, five New Zealand white rabbits (mean weight 4.8±0.2 Kg) were investigated on five different days at least four weeks apart, using a protocol described previously (Gu et al., 1999). Each rabbit received a EP 40 mg sublingual tablet from each formulation set forth in Table XII and a EP 0.3 mg IM in the right thigh from EpiPen.

After EP IM, the remaining EpiPen auto-injector content was evacuated into test tube, sealed and frozen at −20° C. until analyzed for EP content using a reverse phase high performance liquid chromatography (HPLC) system (Waters Corp., Milford, Mass.) with ultra violet detection (UV).

### (h) Example 8 Measurement of Plasma Epinephrine Concentrations

An indwelling catheter (OPTIVA 22G 1″, Johnson & Johnson) was inserted into an ear artery 30 min before dosing. A 2 ml blood sample was obtained immediately before dosing and at 5, 10, 15, 20, 30, 40, 60, 90, 120, 150 and 180 minutes afterward.

Blood samples were refrigerated within 1 hour of sampling and centrifuged at 4° C. Plasma was frozen at −20° C. Before analysis, the plasma was thawed at room temperature and EP was extracted by a solid-liquid extraction process, with an efficiency of 70%-80%. Epinephrine concentrations were measured using HPLC system with electrochemical detection (EC) (Hjemdahl, 1984, Acta Physiol Scand Suppl 527: 43-54; Hjemdahl, 1987, Methods Enzymol 142: 521-534; Ganhao et al., 1991, J Chromatogr 564: 55-66). Two calibration curves with two different epinephrine concentrations ranges were prepared. The low range calibration curve was linear over the range of 0.1 to 1.0 ng/ml with a coefficient of variation of 0.8% at 0.1 ng and 1.4% at 1.0 ng. The high range calibration curve was linear over the range of 1.0 to 10.0 ng/ml with a coefficient of variation of 4.8% at 1.0 ng and 1.1% at 10.0 ng.

### (i) Example 9

### Data Analysis

The maximum plasma EP concentration (Cₘₐₓ), the time at which Cₘₐₓ was achieved (Tₘₐₓ), and the area under the plasma concentration versus time curves (AUC) were calculated from the plasma epinephrine concentration versus time plots using WinNonlin® 5.0 (Pharsight, Mountain View, Calif.). The AUC, Cₘₐₓ, and Tₘₐₓ values for each rabbit were compared using repeated measures ANOVA, Tukey-Kramer tests, and paired Students' t-test using NCSS Statistical Analysis Software (NCSS, Kaysville, Utah). Differences were considered to be significant at p<0.05.

### Results

### (j) Example 10

### In Vitro Results

The powders from all four formulations (Formulations I-D, II-E, III-F, and IV-G) resulted in good mixing, flowability and compression characteristics. The components of each formulation are set forth in Table XII. Tablets from the four formulations met the USP standards for tablet weight variation and content uniformity.

The mean (±SEM) hardness, disintegration time and wetting time results of the four tablet formulations are summarized in Table XIII. Tablet hardness was similar for all four formulations. The disintegration and wetting times were less than 15 sec and 60 min, respectively, for all the four tablet formulations. Tablets from formulation D and E met the USP standards for tablet friability.

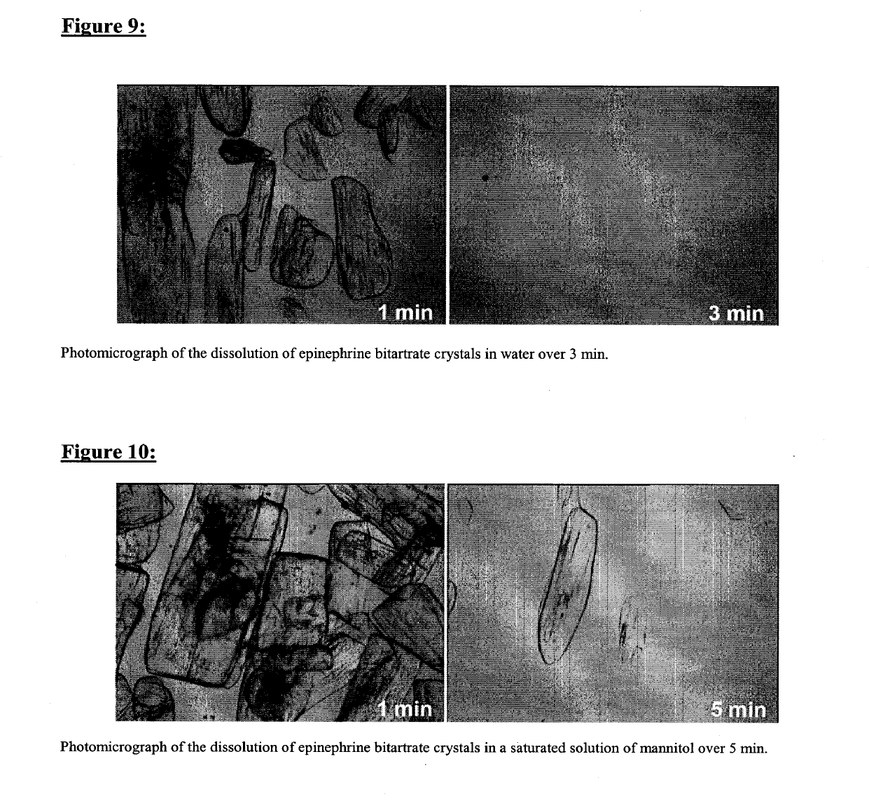

The dissolution of 7.3 mg of EPBT in 100 μl of a saturated solution of mannitol was incomplete after 5 minutes (FIG. 10) in comparison to the dissolution in 100 μl of water, control, which was complete within 3 minutes (FIG. 9).

### (k) Example 11

### In Vivo Results

The mean (±SEM) EP dose injected using EpiPen auto-injectors was 0.34±0.002 mg, calculated by multiplying the EP concentration, measured in the evacuated EpiPen solutions by the stated injected volume (0.3 ml).

Mean (±SEM) plasma EP concentration versus time plots after the administration of EP 40 mg sublingual tablets of each formulation and EP 0.3 mg IM are shown in FIG. 8. Mean (±SEM), AUC, C_{baseline} (endogenous), Cₘₐₓ, and Tₘₐₓ values after the administration of EP 40 mg sublingual tablets of each formulation and EP 0.3 mg IM are shown in Table XIV. No adverse effects were observed.

As shown in Table XIV, the mean (±SEM) AUC after the administration of EP 40 mg sublingual tablets of formulation I-D (1861±537 ng/ml/min) and EP 0.3 mg IM (2431±386 ng/ml/min) did not differ significantly. Mean AUC after the administration of EP 40 mg of formulation II-E (615±87 ng/ml/min), formulation IV-G (606±149 ng/ml/min), and formulation III-F (646±202 ng/ml/min) sublingual tablets were significantly lower than after EP 0.3 mg IM (2431±386 ng/ml/min).

Table XIV also provides that the mean (±SEM) Cₘₐₓ values after EP 40 mg sublingual tablets of formulation I-D (31.0±13.1 ng/ml) and EP 0.3 mg IM (50.3±17.1 ng/ml) did not differ significantly. Mean Cₘₐₓ values after EP 40 mg of formulation II-E (6.0±0.9 ng/ml), formulation IV-G (7.1±1.6 ng/ml), and formulation III-F (6.7±3.2 ng/ml) sublingual tablets were significantly lower than after EP 0.3 mg IM (50.3±17.1 ng/ml).

In addition, Table XIV shows that the mean (±SEM) Tₘₐₓ after the administration of EP 40 mg of formulation I-D (9±4 min), formulation II-E (28±10 min), formulation IV-G (27±9 min), and formulation III-F (16±4 min) sublingual tablets, and EP 0.3 mg IM (21±11 min) did not differ significantly.

As will be appreciated by one of skill in the art, in comparison to the limited range of doses currently available in auto-injectors, buccal and sublingual tablets can be formulated in a wide range of EP doses to provide accurate doses for individuals over a wide range of ages and respective body weights and condition. The tablets are easy to carry and unobtrusive to self-administer and multiple dosing becomes readily available.

The bioavailability of EP following the buccal or sublingual administration of, for example, a 40 mg dose from different fast-disintegrating tablet formulations may vary considerably as a result of differing non-medicinal ingredients content. Even though only tablets from formulations I-D and IV-G passed the USP friability test, tablets from formulations II-E and III-F were hard enough for sublingual administration. The four tablet formulations (Formulations I-D, II-E, III-F, and IV-G) resulted in similar tablet disintegration and wetting times and met the USP tablet content and weight variation (Table X and Table XIII). However, only formulation I-D resulted in AUC, Cₘₐₓ and Tₘₐₓ values that did not differ from those following a mean dose of 0.34 mg EP IM.

The difference between the four tablet formulations (Formulations I-D, II-E, III-F, and IV-G) is affected by the type of excipients used in these formulations (Table XII). The rate-limiting step for EP absorption following sublingual administration is the rate of dissolution. The EP salt used in these tablet formulations, EPBT, is highly water-soluble (1 gm in 3 ml water). However, the rate of dissolution of EPBT can be influenced by the presence of other water-soluble excipients. Mannitol, a highly water-soluble excipient (1 gm in 5.5 ml water) was used in formulations II-E and IV-G, 24.74% and 26.0% of the tablet weight respectively. The dissolution of crystalline EPBT in water occurred rapidly and was complete in less than 3 minutes (as shown in FIG. 9). However, its dissolution in a saturated solution of mannitol was slow and incomplete at the end of 5 minutes (FIG. 10), the length of time at which the tablet was held under the rabbit's tongue. Similarly, mannitol in formulations II-E and IV-G can reduce the dissolution rate and extent of EPBT, especially in the limited saliva volumes available in the sublingual cavity, and therefore can reduce EP bioavailability.

The AUC and Cₘₐₓ values following the 40 mg sublingual EP dose in formulations II-E, III-F and IV-G are significantly lower than those following a mean dose of 0.34 mg EP IM. This reduction in the AUC and Cₘₐₓ values indicates that EPBT dissolution was reduced by mannitol in formulations II-E and IV-G. Formulation III-F was formulated using Pharmaburst®.

It is unlikely that EP absorption from the saliva into the blood circulation across the sublingual epithelial mucosa is influenced by any of the excipients used in these four formulations (Formulations I-D, II-E, III-F, and IV-G). Monosaccharides are absorbed by a secondary active transport utilizing Na cotransporters (The Digestive System. In Human Physiology: From Cells to Systems, Sherwood L. (ed). Brooks/Cole-Thomson Learning: Belmont Calif., 2004; pp 591-645) and should not be interfering with the EP transcellular passive absorption. Water-insoluble excipients are not absorbable because they do not dissolve in the saliva.

Thus, formulations containing a substantial amount of a highly water-soluble excipient, such as mannitol, reduce the dissolution of EP salt and therefore the bioavailability of EP. As discussed above, the sublingual administration of 40 mg of EP from a water-insoluble, rapidly disintegrating tablet resulted in plasma EP concentrations similar to those obtained after EP 0.34 mg IM injection in the thigh.

Thus, in certain embodiments, certain components, such as for example calcium sulfate may be unsuitable for use as a diluent in the invention, as would be any diluent that was virtually insoluble and not suitable for direct-compression techniques, as these would require “wet” granulation first which would affect epinephrine stability.

Conversely, diluents such as for example, lactose, mannitol, sodium chloride, dry starch, and powdered sugar are very soluble. However, in certain embodiments, such diluents may not be optimal because of the possibility that they could compete with epinephrine to dissolve in saliva.

Furthermore, as will be apparent to one of skill in the art, compressed tablets that permit disintegration in the mouth by chewing are not suitable for epinephrine delivery. Medications in the oral cavity, after chewing, could be absorbed in the oral cavity, but would mainly be swallowed for oral absorption. Most of these diluents are not suitable for direct-compression methods, thus moisture would be used in a wet granulation. These ingredients are very water soluble and would compete with epinephrine for dissolution and absorption. If epinephrine is swallowed, it is metabolized in the gut to inactive compounds.

We have determined that it is very important to use water-insoluble ingredients.

It has been shown that the presence of highly water-soluble diluents such as mannitol, mannose, dextrose, sucrose and other and any other “sugars,” may compete for solubility with the epinephrine in the small volume of saliva in the sublingual cavity.

A number of the above “sugars” are also used in the so-called “fast-melt” type of formulation. This type of formulation would not be suitable for sublingual epinephrine for two reasons: (1) Formulation of fast-melt type of products usually involves preparing a solution of the fast-melt ingredients with the active medication, e.g. epinephrine. Water is removed to leave a type of fast melt gum or gel. Water should not be used in the formulation of epinephrine sublingual formulations because epinephrine could decompose in the aqueous environment before the water is removed; and (2) The presence of high concentrations of highly water soluble materials would compete with the epinephrine to dissolve in the small sublingual volume of saliva.

If the epinephrine from the sublingual tablet does not dissolve rapidly or optimally in the saliva, then sublingual absorption is impeded or even inhibited. This information is supported by our studies of the “other” 40 mg tablets tested by our research group and included in our examples disclosed herein (Formulations II-E, 111-F and IV-G).

The tablet formulations described herein, e.g., Formulations I-A, I-B, I-C, I-D (Table VI) overcome the situation described in #1 in that the primary excipient is the insoluble microcrystalline cellulose (MCC).

Analysis of Long Term Stability of Fast Disintegrating Sublingual Epinephrine Tablets

### (l) Example 12

### Materials

(−)-Epinephrine (+) bitartrate, (−)-3,4-dihydroxy-α[(methylamino)methyl]benzyl alcohol (+)-tartrate (1:1) salt, was purchased from Sigma-Aldrich (St. Louis, Mo.). Ceolus® PH-301 (microcrystalline cellulose) with a mean particle size of 50 μm was supplied by Asahi Kasei Chemicals Corp. (Tokyo, Japan) and low-substituted hydroxypropyl cellulose (LH11) with a mean particle size of 50 μm was supplied by Shin-Etsu Chemical Co (Tokyo, Japan). Magnesium stearate was purchased from Mallinckrodt Baker (Phillipsburg, N.J.).

### (m) Example 13

### Preparation and Evaluation of Tablets

Three fast-disintegrating tablet batches containing 10 mg (Formulation I-B), 20 mg (Formulation I-C), and 40 mg (Formulation I-D) of epinephrine were manufactured by the direct compression methods described herein. These tablets were formulated using microcrystalline cellulose, low-substituted hydroxylpropyl cellulose, and magnesium stearate. The tablet weight was 150 mg. All excipients were kept under low humidity condition before mixing. The mixing process was performed in a light-resistant container after flushing the container with nitrogen. The prepared powder mixture of the three tablet batches were compressed directly after mixing at a pre-selected compression force for each tablet batch that permits rapid tablet disintegration and wetting while retaining sufficient hardness to withstand shipping and handling. All batches were tested for tablet weight variation and drug content uniformity using the USP methods and criteria as described above. A tablet from each tablet batch was dissolved in 2.0 mL solvent of 0.1 M perchloric acid and 0.1 mM sodium metabisulfite. An aliquot of 50 μL was withdrawn and diluted to 2.0 mL with the solvent. Drug content was analyzed using high performance liquid chromatography system with ultra violet detection (Waters Corp., Milford, Mass.).

### (n) Example 14

### Storage of Tablets

Each of the three tablet batches was divided into three equal portions and immediately transferred into tightly closed, opaque, plastic tablet containers with desiccants. Container 1 was stored at 25° C. (room temperature), container 2 was stored at 5° C. (refrigerator), and container 3 was flushed with nitrogen before being tightly closed and stored at 5° C. From the three containers of the 10 mg and 20 mg epinephrine tablet batches six-tablet samples, randomly selected, were withdrawn at six and twelve months. Containers stored under nitrogen were re-flushed with nitrogen before being sealed and stored for the next time period. From the three containers of the 40 mg epinephrine tablet batch six-tablet samples, randomly selected, were withdrawn at twenty months. The six tablets in each sample were observed immediately for any visual changes, and then dissolved and diluted to be analyzed for epinephrine content. The mean±standard error (SEM) epinephrine dose data obtained from the content uniformity test for each tablet batch were used as the control epinephrine content values at baseline before storage commenced.

### (o) Example 15

### Data Analysis

For each tablet batch, the epinephrine dose remaining in the tablets selected from the three containers, stored under the three different storage conditions, for different storage periods were calculated and compared with each other and with control using Two-Way ANOVA and Tukey-Kramer tests using NCSS Statistical Analysis Software (NCSS, Kaysville, Utah). Differences were considered to be significant at p<0.05.

### (p) Example 16

### Long Term Stability Results

All the three tablet batches were within USP specifications for weight variation and drug content uniformity.

There were no detectable color changes in the 10 mg and 20 mg epinephrine tablet batches stored for six and twelve months under the three storage conditions. Also, there were no detectable color changes in the 40 mg epinephrine tablet batch stored for twenty months at 5° C. with and without nitrogen flushing prior to storage. Slight tablet discoloration was observed in the 40 mg epinephrine tablet batch stored for twenty months at 25° C.

Mean (±SEM) epinephrine doses remaining in the 10 mg and 20 mg epinephrine tablet batches stored for six and twelve months, and in 40 mg epinephrine tablet batch stored for twenty months at 25° C., 5° C., and 5° C. with nitrogen flushing are reported in Table XV.

For the 10 mg epinephrine tablet batch, the mean (±SEM) epinephrine dose remaining in tablets stored for six months at 25° C. (9.2±0.1 mg), 5° C. (9.3±0.2 mg), and at 5° C. with nitrogen flushing (9.4±0.3 mg) and for twelve months at 25° C. (9.6±0.1 mg), 5° C. (9.7±0.2 mg), and at 5° C. with nitrogen flushing (9.6±0.1 mg) did not differ significantly from each other and from the control (9.8±0.1 mg).

For the 20 mg epinephrine tablet batch, the mean (±SEM) epinephrine dose remaining in tablets stored for six months at 25° C. (19.8±0.5 mg), 5° C. (19.8±0.5 mg), and at 5° C. with nitrogen flushing (20.3±0.3 mg) and for twelve months at 25° C. (19.4±0.4 mg), 5° C. (20.3±0.3 mg), and at 5° C. with nitrogen flushing (20.9±0.8 mg) did not differ significantly from each other and from the control (20.1±0.3 mg).

For the 40 mg epinephrine tablet batch, the mean (±SEM) epinephrine dose remaining in tablets stored for twenty months at 25° C. (37.5±0.2 mg), 5° C. (38.9±0.6 mg), and at 5° C. with nitrogen flushing (38.5±1.2 mg) did not differ significantly from each other and from the control (38.0±0.6 mg).

These fast-disintegrating epinephrine tablets were stable for twelve months under the three storage conditions, 25° C., 5° C., and 5° C. with nitrogen flushing. They were stable for twenty months at 5° C. with and without nitrogen flushing. The epinephrine dose remaining in the 40 mg epinephrine tablets stored for twenty months at 25° C. did not differ significantly from control and from tablets stored at 5° C. with and without nitrogen flushing. These results showed that the use of opaque containers to reduce light, desiccants to reduce humidity in the container, and low temperatures prevented tablet discoloration for at least twenty months. Exposing the tablets to oxygen at 25° C., 5° C. did not affect the stability of epinephrine since flushing the container with nitrogen prior storage at 5° C. did not result in significantly higher epinephrine content in these tablets.

Since epinephrine is a very labile compound and can be decomposed by heat, light and air (oxygen) we considered the reason for the long term stability demonstrated by these results. While not wishing to be bound to a specific theory, we believe that possible explanations for the long-term stability may be that (i) moisture exposure was minimized during production of the formulations and/or (ii) MCC contains the lowest percentage of hydroperoxides (HPO) of any of the insoluble excipients used in our formulations. These peroxides, if present could hasten the decomposition (oxidation) of epinephrine in these sublingual tablets. HPO has been shown to be present in concentrations<10 nmole HPO/g in MCC. Other insoluble excipients, such as the polymer povidone, have been shown to contain HPO levels of 20,000 nmoles HPO/g.

Soluble excipients such as lactose, sucrose and mannitol have been shown to contain low levels of HPO, but these are soluble. Thus, in some embodiments, these excipients may not be optimal because of the possibility of the problem described in #1 above.

Slightly soluble excipients such as polysorbate 80, polyethylene glycol 400 have been found to contain considerable concentrations of HPO. Thus, in some embodiments, these excipients may not be optimal because of the possibility because of solubility and HPO complications.

In certain instances, insoluble excipients such as povidone and hydroxypropyl cellulose may contain very high concentrations of HPO. Thus, in some embodiments, these excipients may not be optimal because of the possibility of HPO complications.

While the preferred embodiments of the invention have been described above, it will be recognized and understood that various modifications may be made therein, and the appended claims are intended to cover all such modifications which may fall within the spirit and scope of the invention.

TABLE I
Composition of Four Tablet Formulations of Epinephrine*

| | Tablet Formulations | | | |
|---|---|---|---|---|
| Ingredient % | A | B | C | D |
| Epinephrine Bitartrate | — | 6 | 12 | 24 |
| Microcrystalline Cellulose (PH-301) | 88.2 | 82.8 | 77.4 | 66.6 |
| Low-Substituted Hydroxypropyl | 9.8 | 9.2 | 8.6 | 7.4 |
| Cellulose (LH11) | | | | |
| Magnesium Stearate | 2 | 2 | 2 | 2 |

*Tablet weight was 150 mg.

TABLE II
The Effect of Increasing Compression Force (CF) on the Tablet Hardness (H)

| | A | | B | | C | | D | |
|---|---|---|---|---|---|---|---|---|
| CF (KN) | H^{a} (KgF) | CV % | H^{a} (KgF) | CV % | H^{a} (KgF) | CV % | H^{a} (KgF) | CV % |
| 21.5 | 1.9 ± 0.1 | 4.5 | — | — | — | — | — | — |
| 22.0 | 2.5 ± 0.2 | 6.6 | — | — | — | — | — | — |
| 22.5 | 3.6 ± 0.2 | 5.9 | 1.8 ± 0.1 | 4.7 | — | — | — | — |
| 23.0 | 4.7 ± 0.4 | 8.5 | 2.5 ± 0.2 | 7.3 | — | — | — | — |
| 23.5 | 7.2 ± 0.3 | 4.5 | 4.1 ± 0.2 | 5.1 | 1.5 ± 0.1 | 5.5 | 1.2 ± 0.1 | 9.8 |
| 24.0 | 12.0 ± 0.4 | 3.0 | 4.9 ± 0.6 | 11.4 | 2.3 ± 0.2 | 10 | 2.0 ± 0.2 | 8.1 |
| 24.5 | — | — | 10.3 ± 0.5 | 4.6 | 4.0 ± 0.3 | 7.7 | 3.1 ± 0.2 | 6.6 |
| 25.0 | — | — | — | — | 6.5 ± 0.2 | 3.4 | 4.5 ± 0.1 | 2.9 |
| 25.5 | — | — | — | — | 9.0 ± 1.2 | 12.9 | 9.1 ± 0.1 | 1.4 |

^{a}mean ± SD

TABLE III
Correlation Constants, a and b, for the Four Tablet Formulations*

| | A | | B | | C | | D | |
|---|---|---|---|---|---|---|---|---|
| Constants for | a | b | a | b | a | b | a | b |
| H vs. CF | 3 × 10⁻⁰⁷ | 0.72 | 1 × 10⁻⁰⁸ | 0.83 | 7 × 10⁻¹⁰ | 0.92 | 1 × 10⁻¹⁰ | 0.98 |
| DT vs. CF | 63.32^{†} | 3.04^{†} | 4 × 10⁻⁰⁸ | 0.80 | 2 × 10⁻⁰⁷ | 0.72 | 8 × 10⁻¹² | 1.14 |
| WT vs. CF | 67.54^{†} | 3.56^{†} | 1 × 10⁻⁶ | 0.68 | 6 × 10⁻¹⁴ | 1.38 | 2 × 10⁻¹⁴ | 1.44 |
| WT vs. DT^{†} | −1.26 | 2.26 | 2.44 | 2.70 | 26.25 | 7.19 | 4.71 | 3.40 |

*CF indicates compression force (KN); H, tablet hardness (kg); CV, coefficient of variation; DT, disintegration time (sec); WT, wetting time (sec).
^{†}Constants derived using equation 2 (all other constants derived using equation 1).

TABLE IV
The Effect of Increasing Compression Force (CF) on the Tablet Disintegration Time (DT)

| | A | | B | | C | | D | |
|---|---|---|---|---|---|---|---|---|
| CF | DT^{a} | | DT^{a} | | DT^{a} | | DT^{a} | |
| (KN) | (sec) | CV % | (sec) | CV % | (sec) | CV % | (sec) | CV % |
| 21.5 | 2.2 ± 0.4 | 20.3 | — | — | — | — | — | — |
| 22.0 | 3.2 ± 0.4 | 14.0 | — | — | — | — | — | — |
| 22.5 | 5.2 ± 0.4 | 8.6 | 2.8 ± 0.5 | 16.0 | — | — | — | — |
| 23.0 | 6.8 ± 1.5 | 21.8 | 3.8 ± 0.4 | 11.8 | — | — | — | — |
| 23.5 | 8.0 ± 0.7 | 8.8 | 6.2 ± 0.8 | 13.5 | 4.6 ± 0.6 | 11.9 | 4.6 ± 0.6 | 11.9 |
| 24.0 | 37.2 ± 2.2 | 5.8 | 9.0 ± 1.0 | 11.1 | 5.8 ± 0.4 | 7.7 | 5.6 ± 0.5 | 9.8 |
| 24.5 | — | — | 120.0 ± 7.9 | 6.6 | 7.6 ± 0.9 | 11.8 | 9.4 ± 0.9 | 9.5 |
| 25.0 | — | — | — | — | 14.0 ± 1.4 | 10.1 | 26.0 ± 6.4 | 24.8 |
| 25.5 | — | — | — | — | >120 | — | >120 | — |

^{a}mean ± SD

TABLE V
The Effect of Increasing Compression Force (CF) on the Tablet Wetting Time (WT)

| | A | | B | | C | | D | |
|---|---|---|---|---|---|---|---|---|
| CF | WT^{a} | | WT^{a} | | WT^{a} | | WT^{a} | |
| (KN) | (sec) | CV % | (sec) | CV % | (sec) | CV % | (sec) | CV % |
| 21.5 | 8.2 ± 0.4 | 5.5 | — | — | — | — | — | — |
| 22.0 | 11.4 ± 0.9 | 7.8 | — | — | — | — | — | — |
| 22.5 | 13.4 ± 2.3 | 17.2 | 7.2 ± 0.4 | 6.2 | — | — | — | — |
| 23.0 | 14.0 ± 2.0 | 14.3 | 8.8 ± 0.8 | 9.5 | — | — | — | — |
| 23.5 | 15.8 ± 2.2 | 13.7 | 11.0 ± 0.7 | 6.4 | 8.6 ± 0.9 | 10.3 | 9.0 ± 0.7 | 7.9 |
| 24.0 | 86.0 ± 16.1 | 18.7 | 20.8 ± 2.2 | 10.4 | 16.6 ± 1.8 | 10.9 | 16.4 ± 1.1 | 7.0 |
| 24.5 | — | — | 102.4 ± 21.6 | 21.1 | 24.4 ± 1.7 | 6.9 | 27.2 ± 3.4 | 12.6 |
| 25.0 | — | — | — | — | 75.6 ± 11.9 | 15.8 | 83.6 ± 31.7 | 38.0 |
| 25.5 | — | — | — | — | >120 | — | >120 | — |

^{a}mean ± SD

TABLE VI
Formulation I

| | | | |
|---|---|---|---|
| Ingredients | Type | Weight (mg) | Percentage % |

Formulation I-A (Placebo Tablets)

| | | | | |
|---|---|---|---|---|
| 1 | Active Ingredient | Epinephrine bitartrate | 0 | 0 |
| 2 | Fillers | Microcrystalline cellulose* | 132.3 | 88.2 |
| 3 | Disintegrant | Low-substituted hydroxypropyl | 14.7 | 9.8 |
| | | cellulose** | | |
| 4 | Lubricant | Magnesium Stearate | 3 | 2 |
| | Tablet Weight | | 150 | 100 |

Formulation I-B (10 mg of Epinephrine)

| | | | | |
|---|---|---|---|---|
| 1 | Active Ingredient | Epinephrine bitartrate | 18.193 | 12.13 |
| 2 | Fillers | Microcrystalline cellulose* | 115.927 | 77.28 |
| 3 | Disintegrant | Low-substituted | 12.88 | 8.59 |
| | | hydroxypropyl cellulose** | | |
| 4 | Lubricant | Magnesium Stearate | 3 | 2.00 |
| | Tablet Weight | | 150 | 100 |

Formulation I-C (20 mg of Epinephrine)

| | | | | |
|---|---|---|---|---|
| 1 | Active Ingredient | Epinephrine bitartrate | 36.387 | 24.26 |
| 2 | Fillers | Microcrystalline cellulose | 99.552 | 66.37 |
| 3 | Disintegrant | Low-substituted | 11.061 | 7.37 |
| | | hydroxypropyl cellulose** | | |
| 4 | Lubricant | Magnesium Stearate | 3 | 2.00 |
| | Tablet Weight | | 150 | 100 |

Formulation I-D (40 mg of Epinephrine)

| | | | | |
|---|---|---|---|---|
| 1 | Active Ingredient | Epinephrine bitartrate | 72.768 | 48.51 |
| 2 | Fillers | Microcrystalline cellulose* | 66.809 | 44.54 |
| 3 | Disintegrant | Low-substituted | 7.423 | 4.95 |
| | | hydroxypropyl cellulose** | | |
| 4 | Lubricant | Magnesium Stearate | 3 | 2.00 |
| | Tablet Weight | | 150 | 100 |

*Ceolus ®-PH-301 (50 μm);
**L-HPC-LH11

TABLE VII
Formulation II
Formulation II-E (40 mg of Epinephrine)

| | Ingredients | Type | Weight (mg) | Percentage % |
|---|---|---|---|---|
| 1 | Active Ingredient | Epinephrine bitartrate | 72.768 | 48.51 |
| 2 | Fillers | Microcrystalline cellulose* | 33.404 | 22.27 |
| 3 | Fillers | Manitol** | 37.116 | 24.74 |
| 4 | Disintegrant | Low-substituted hydroxypropyl | 3.712 | 2.47 |
| | | cellulose^{¥} | | |
| 5 | Lubricant | Magnesium Stearate | 3 | 2.00 |
| | Tablet Weight | | 150 | 100 |

*Ceolus ®-PH-M-06 (7 μm);
**Pearlitol ® 400 DC;
^{¥}L-HPC-LH11

TABLE VIII
Formulation III
Formulation III-F (40 mg of Epinephrine)

| | Ingredients | Type | Weight (mg) | Percentage % |
|---|---|---|---|---|
| 1 | Active | Epinephrine | 72.768 | 48.51 |
| | Ingredient | bitartrate | | |
| 2 | Fillers | Pharmaburst ® | 74.232 | 49.49 |
| | | -C1 | | |
| 3 | Lubricant | Sodium stearyl | 3 | 2.00 |
| | | fumarate* | | |
| | Tablet Weight | | 150 | 100 |

*PRUV ®

TABLE IX
Formulation IV
Formulation IV-G (40 mg of Epinephrine)

| | Ingredients | Type | Weight (mg) | Percentage % |
|---|---|---|---|---|
| 1 | Active Ingredient | Epinephrine bitartrate | 72.77 | 48.51 |
| 2 | Fillers | Calcium silicate* | 15.83 | 10.55 |
| 3 | Fillers | Microcrystalline cellulose** | 19.31 | 12.87 |
| 4 | Fillers | Manitol^{§} | 39.00 | 26.00 |
| 5 | Disintegrant | Crospovidone^{¥} | 1.95 | 1.30 |
| 6 | Glidant | Silicon dioxide^{£} | 0.39 | 0.26 |
| 7 | Lubricant | Magnesium Stearate | 0.77 | 0.51 |
| | Tablet Weight | | 150 | 100.00 |

*Rxcipients ® FM1000;
**Ceolus ® KG-802 (50 μm);
^{§}Pearlitol ®;
^{¥}Polyplasdone ® XL-10;
^{£}Rxcipients ® GL200

TABLE X
In Vitro Data: for all the formulations used in animal studies*

| | | | | | WV | CV | Friability |
|---|---|---|---|---|---|---|---|
| Formulations | CF (KN) | H (Kgf) | DT (sec) | WT (sec) | (C of V %) | (C of V %) | (%) |
| I-A | 22.5 | 3.6 ± 0.1 | 5.2 ± 0.2 | 13.4 ± 1.0 | — | — | 0.08 |
| I-B | 22.5 | 3.1 ± 0.1 | 7.7 ± 0.3 | 24.2 ± 0.9 | 1.8 | 3.3 | 0.17 |
| I-C | 23 | 2.9 ± 0.1 | 12.0 ± 0.6 | 41.8 ± 3.6 | 2.1 | 3.6 | 0.36 |
| I-D | 24 | 2.4 ± 0.1 | 13.5 ± 0.2 | 26.2 ± 1.8 | 2.2 | 4.8 | 0.62 |
| II-E | 19.5 | 1.5 ± 0.1 | 13.2 ± 0.8 | 47.3 ± 3.3 | 0.8 | 2.4 | 13.4 |
| III-F | 19.5 | 2.6 ± 0.1 | 8.3 ± 0.3 | 26.5 ± 2.0 | 1.5 | 2.2 | 6.5 |
| IV-G | 17.5 | 2.4 ± 0.1 | 9.3 ± 0.5 | 14.3 ± 0.6 | 0.5 | 2.3 | 0.33 |

CF: Compression Force;
H: Hardness;
DT: Disintegration Time;
WT: Wetting Time;
WV: Tablets Weight Variation;
CV: Tablets Content Variation;
C of V: Coefficient of Variation
*Data are represented as mean ± SE

TABLE XI
In Vivo Data: for all the formulations and EpiPen ®
used in animal studies*

| Formulations | AUC (ng/ml/min) | Cmax (ng/ml) | Tmax (min) |
|---|---|---|---|
| I-A | 472 ± 126 | 6.5 ± 1.3 | — |
| I-B | 335 ± 152 | 5.2 ± 2.3 | 37 ± 11 |
| I-C | 801 ± 160 | 6.6 ± 1.4 | 31 ± 9 |
| I-D | 1861 ± 537 | 31.0 ± 13.1 | 9 ± 2 |
| EpiPen ®** | 2431 ± 386 | 50.3 ± 17.1 | 21 ± 5 |
| II-E | 615 ± 87 | 6.0 ± 0.9 | 28 ± 10 |
| III-F | 606 ± 149 | 7.1 ± 1.6 | 27 ± 9 |
| IV-G | 646 ± 202 | 6.7 ± 3.2 | 16 ± 4 |

AUC: Area Under the Curve;
Cmax: the maximum concentration;
Tmax: time at maximum concentration
*Data are represented as mean ± SE
**EpiPen ® (0.3 mg) - EpiPen ® is an autoinjector that delivers 0.3 mg of epinephrine. EpiPen ® is manufactured by EM Industries, Inc and marketed in Canada by Allerex Lab. Ltd. (lot # 4C6361). EpiPen ® is injected in the thigh of rabbit model.

TABLE XII
Composition of four tablet formulations of epinephrine*

| | Tablet Formulations | | | |
|---|---|---|---|---|
| Ingredient % | I-D | II-E | IV-G | III-F |
| Epinephrine Bitartrate | 48.51 | 48.51 | 48.51 | 48.51 |
| Microcrystalline Cellulose (PH-301) | 44.54 | — | — | — |
| Microcrystalline Cellulose (PH-M-06) | — | 22.27 | — | — |
| Microcrystalline Cellulose (KG-802) | — | — | 12.87 | — |
| Calcium Silicate | — | — | 10.55 | |
| Pharmaburst ® | — | — | — | 49.49 |
| Low-Substituted Hydroxypropyl Cellulose | 4.95 | 2.47 | — | — |
| (LH11) | | | | |
| Crospovidone | | — | 1.3 | — |
| Mannitol | — | 24.74 | 26.00 | — |
| Silicon Dioxide | — | — | 0.26 | — |
| Magnesium Stearate | 2.00 | 2.00 | 0.51 | — |
| Sodium Stearyl Fumerate | — | — | — | 2.00 |

*Tablet weight was 150 mg.

TABLE XIII
The hardness, disintegration time, wetting time, and
friability of four tablet formulations*^{†}

| Formu- | In Vitro Tablets Characteristics | | | | | | |
|---|---|---|---|---|---|---|---|
| lations | H | CV | DT | CV | WT | CV | F |
| I-D | 2.4 ± 0.1 | 12.4 | 13.5 ± 0.2 | 4.1 | 26.2 ± 1.8 | 17.0 | 0.6 |
| II-E | 1.5 ± 0.1 | 16.9 | 13.2 ± 0.8 | 14.7 | 47.3 ± 3.3 | 16.9 | 13.4 |
| IV-G | 2.4 ± 0.1 | 7.5 | 9.3 ± 0.5 | 13.0 | 14.3 ± 0.6 | 9.5 | 0.3 |
| III-F | 2.6 ± 0.1 | 4.3 | 8.3 ± 0.3 | 9.8 | 26.5 ± 2.0 | 18.2 | 6.5 |

*mean ± SEM (n = 6).
^{†}H indicates tablet hardness (kg); CV, coefficient of variation (%); DT, disintegration time (sec); WT, wetting time (sec); F: friability (%) (USP limits ≦1%).

TABLE XIV
Epinephrine bioavailability after 40 mg sublingual epinephrine administration from four different
tablet formulations and epinephrine 0.3 mg intramuscular (IM) injection in the thigh

| | Sublingual Tablets | | | | IM Injection |
|---|---|---|---|---|---|
| Mean ± SEM* | I-D | II-E | IV-G | III-F | EpiPen ® |
| Epinephrine | 38.15 ± 0.51 | 35.79 ± 0.30 | 39.20 ± 0.29 | 39.34 ± 0.28 | 0.34 |
| dose (mg) | | | | | |
| AUC_{0-3 h} | 1861 ± 537 | 615 ± 87^{†} | 606 ± 149^{†} | 646 ± 202^{†} | 2431 ± 386 |
| (ng/mL/min) | | | | | |
| C_{baseline} (ng/mL) | 15.4 ± 3.2 | 4.2 ± 0.7 | 11.2 ± 7.5 | 3.5 ± 1.4 | 9.6 ± 3.5 |
| Cₘₐₓ (ng/mL) | 31.0 ± 13.0 | 6.0 ± 0.9^{†} | 7.1 ± 1.6^{†} | 6.7 ± 3.2^{†} | 50.0 ± 17.0 |
| Tₘₐₓ (min) | 9 ± 2 | 28 ± 10 | 27 ± 9 | 16 ± 4 | 21 ± 5 |

*n = 5.
^{†}p < 0.05 compared to intramuscular (IM) injection.
AUC: area under the plasma concentration versus time curve;
C_{baseline}: Baseline plasma concentration (endogenous epinephrine);
Cₘₐₓ: maximum plasma concentration (mean ± SEM of individual Cₘₐₓ values from each rabbit, regardless of the time at which Cₘₐₓ was achieved);
Tₘₐₓ: time at which maximum plasma epinephrine concentration was achieved (mean ± SEM of individual Tₘₐₓ values from each rabbit).

TABLE XV
Epinephrine doses remaining in 10 mg, 20 mg, and 40 mg epinephrine
tablet batches stored at 25° C., 5° C., and 5° C. with
nitrogen flushing (5° C.-N₂) for six, twelve, and twenty months.^{a}

| | | | | | 40 mg |
|---|---|---|---|---|---|
| | 10 mg epinephrine | | 20 mg epinephrine | | epinephrine |
| Storage | tablets^{b} | | tablets^{c} | | tablets^{d} |
| condition | 6 Months | 12 Months | 6 Months | 12 Months | 20 Months |
| 25° C. | 9.2 ± 0.1 | 9.6 ± 0.1 | 19.8 ± 0.5 | 19.4 ± 0.4 | 37.5 ± 0.2 |
| 5° C. | 9.3 ± 0.2 | 9.7 ± 0.2 | 19.8 ± 0.5 | 20.3 ± 0.3 | 38.9 ± 0.6 |
| 5° C.-N₂ | 9.4 ± 0.3 | 9.6 ± 0.1 | 20.3 ± 0.3 | 20.9 ± 0.8 | 38.5 ± 1.2 |

^{a}mean ± SEM (n = 6).
^{b}epinephrine dose in the control tables was 9.8 ± 0.1 mg.
^{c}epinephrine dose in the control tablets was 20.1 ± 0.3 mg.
^{d}epinephrine dose in the control tables was 38.0 ± 0.6 mg.

What is claimed is:

1. A pharmaceutical tablet for sublingual application comprising:
about 48.5% epinephrine (EPBT);
about 44.5% microcrystalline cellulose;
about 5% low-substituted hydroxypropyl cellulose; and
about 2% Magnesium stearate.

2. A pharmaceutical tablet for sublingual application comprising:
about 24.3% epinephrine (EPBT);
about 66.4% microcrystalline cellulose;
about 7.4% low-substituted hydroxypropyl cellulose; and
about 2% Magnesium stearate.

3. A pharmaceutical tablet for sublingual application comprising:
about 12.1% epinephrine (EPBT);
about 77.3% microcrystalline cellulose;
about 8.6% low-substituted hydroxypropyl cellulose; and
about 2% Magnesium stearate.

4. A pharmaceutical tablet for buccal or sublingual administration comprising:
(a) about 0.5% to about 90% epinephrine;
(b) about 7.5% to about 95% filler; and
(c) about 2.5% to about 10.5% disintegrant.

5. The pharmaceutical tablet of claim 4, wherein said pharmaceutical tablet for buccal or sublingual administration comprises:
(a) about 43.5% to about 53.5% epinephrine;
(b) about 39.5% to about 49.5% filler; and
(c) about 2.6% to about 7.0% disintegrant.

6. The pharmaceutical tablet of claim 4, wherein said pharmaceutical tablet for buccal or sublingual administration comprises:
(a) about 19.3% to about 29.3% epinephrine;
(b) about 61.5% to about 71.4% filler; and
(c) about 6.8% to about 9.2% disintegrant.

7. The pharmaceutical tablet of claim 4, wherein said pharmaceutical tablet for buccal or sublingual administration comprises:
(a) about 7.1% to about 17.1% epinephrine;
(b) about 72.4% to about 82.3% filler; and
(c) about 7.9% to about 10.5% disintegrant.

8. The pharmaceutical tablet of claim 4, wherein said buccal or sublingual tablet comprises about 35% to about 85% epinephrine.

9. The pharmaceutical tablet of claim 4, wherein said buccal or sublingual tablet comprises about 40% to about 70% epinephrine.

10. The pharmaceutical tablet of claim 4, wherein said buccal or sublingual tablet comprises about 40% to about 55% epinephrine.

11. The pharmaceutical tablet of claim 4, wherein said buccal or sublingual tablet comprises about 65% to about 90% epinephrine.

12. The pharmaceutical tablet of claim 4, wherein said buccal or sublingual tablet comprises about 35% to about 45% epinephrine.

13. The pharmaceutical tablet of claim 4, wherein said buccal or sublingual tablet comprises about 20% to about 35% epinephrine.

14. The pharmaceutical tablet of claim 4, wherein said buccal or sublingual tablet comprises about 10% to about 15% epinephrine.

15. The pharmaceutical tablet of claim 4, wherein said buccal or sublingual tablet comprises about 2% to about 8% epinephrine.

16. The pharmaceutical tablet of claim 4, wherein said buccal or sublingual tablet comprises about 25 mg to about 75 mg of epinephrine.

17. The pharmaceutical tablet of claim 4, wherein said buccal or sublingual tablet comprises about 35 mg to about 60 mg of epinephrine.

18. The pharmaceutical tablet of claim 4, wherein said buccal or sublingual tablet comprises about 35 mg to about 45 mg of epinephrine.

19. The pharmaceutical tablet of claim 4, wherein said buccal or sublingual tablet comprises about 55 mg to about 75 mg of epinephrine.

20. The pharmaceutical tablet of claim 4, wherein said buccal or sublingual tablet comprises about 25 mg to about 40 mg of epinephrine.

21. The pharmaceutical tablet of claim 4, wherein said buccal or sublingual tablet comprises about 10 mg to about 25 mg of epinephrine.

22. The pharmaceutical tablet of claim 4, wherein said buccal or sublingual tablet comprises about 5 mg to about 10 mg of epinephrine.

23. The pharmaceutical tablet of claim 4, wherein said buccal or sublingual tablet comprises about 0.5 mg to about 5 mg of epinephrine.

24. The pharmaceutical tablet of claim 4, wherein said epinephrine is selected from the group consisting of: racemic mixtures of epinephrine, free base epinephrine, epinephrine bitartrate (EPBT), or epinephrine HCl.

25. The pharmaceutical tablet of claim 4, wherein said filler is selected from the group consisting of: a microcrystalline cellulose, lactose, calcium carbonate, calcium bicarbonate, calcium phosphate, dibasic calcium phosphate, calcium sulfate, calcium silicate, cellulose powders, dextrose, dextrates, dextrans, starches, pregelatinized starches, sucrose, xylitol, lactitol, sorbitol, sodium bicarbonate, sodium chloride, polyethylene glycol, or combinations thereof.

26. The pharmaceutical tablet of claim 25, wherein said filler is a microcrystalline cellulose having a particle size ranging from about 5 μm to about 500 μm.

27. The pharmaceutical tablet of claim 4, wherein said disintegrant is selected from the group consisting of: low-substituted hydroxypropyl celluloses, cross-linked celluloses, cross-linked sodium carboxymethyl celluloses, cross-linked carboxymethyl celluloses, cross-linked croscarmelloses, cross-linked starches, sodium starch glycolate, crospovidone, or combinations thereof.

28. The pharmaceutical tablet of claim 4, wherein said filler is a microcrystalline cellulose and said disintegrant is a low-substituted hydroxypropyl cellulose.

29. The pharmaceutical tablet of claim 4, further comprising a pharmaceutically acceptable excipient.

30. The pharmaceutical tablet of claim 29, wherein said pharmaceutically acceptable excipient is selected from the group consisting of: diluents, binders, glidants, lubricants, colorants, flavorants, coating materials, or combinations thereof.

31. The pharmaceutical tablet of claim 4, wherein the decrease in content of epinephrine after being stored at 25° C. for at least twelve months is less than about 2.5 percent.

32. The pharmaceutical tablet of claim 31, wherein said pharmaceutical tablet comprise from about 10 mg epinephrine to about 40 mg epinephrine.

33. The pharmaceutical tablet of claim 4, wherein the decrease in content of epinephrine after being stored at 5° C. for at least twelve months is less than about 2.5 percent.

34. The pharmaceutical tablet of claim 33, wherein said pharmaceutical tablet comprise from about 10 mg epinephrine to about 40 mg epinephrine.

35. The pharmaceutical tablet of claim 4, wherein the decrease in content of epinephrine after being stored at 5° C. with nitrogen flushing for at least twelve months is less than about 2.5 percent.

36. The pharmaceutical tablet of claim 35, wherein said pharmaceutical tablet comprise from about 10 mg epinephrine to about 40 mg epinephrine.

37. A method of preparing an epinephrine tablet for sublingual administration comprising preparing a mixture of:
(a) about 0.5% to about 90% epinephrine;
(b) about 7.5% to about 95% filler;
(c) about 2.5% to about 10.5% disintegrant; and
(d) compressing a unit dosage portion of the mixture to about 24 kN, thereby producing a tablet.

38. A method of preparing the pharmaceutical tablet for sublingual application of claim 1 comprising preparing a mixture of:
about 48.5% epinephrine (EPBT);
about 44.5% microcrystalline cellulose;
about 5% low-substituted hydroxypropyl cellulose; and
about 2% magnesium stearate; and
compressing a unit dosage portion of the mixture to about 24 kN, thereby producing a tablet.

39. A method of preparing the pharmaceutical tablet for sublingual application of claim 2 comprising preparing a mixture of:
about 24.3% epinephrine (EPBT);
about 66.4% microcrystalline cellulose;
about 7.4% low-substituted hydroxypropyl cellulose; and
about 2% magnesium stearate; and
compressing a unit dosage portion of the mixture to about 24 kN, thereby producing a tablet.

40. A method of preparing the pharmaceutical tablet for sublingual application of claim 3 comprising preparing a mixture of:
about 12.1% epinephrine (EPBT);
about 77.3% microcrystalline cellulose;
about 8.6% low-substituted hydroxypropyl cellulose; and
about 2% magnesium stearate; and
compressing a unit dosage portion of the mixture to about 24 kN, thereby producing a tablet.

41. A method for the treatment of an allergic emergency, comprising the administration of a dose of the pharmaceutical tablet for buccal or sublingual administration of the pharmaceutical tablet of claim 4 to a patient diagnosed with, or suspected of having, an allergic emergency.

42. A method of the treatment of anaphylaxis, comprising the administration of a dose of the pharmaceutical tablet for buccal or sublingual administration of the pharmaceutical tablet of claim 4 to a patient diagnosed with, or suspected of having, anaphylaxis.

43. A method for the treatment of asthma, comprising the administration of a dose of the pharmaceutical tablet for buccal or sublingual administration of the pharmaceutical tablet of claim 4 to a patient diagnosed with, or suspected of having, asthma.

44. A method for the treatment of bronchial asthma, comprising the administration of a dose of the pharmaceutical tablet for buccal or sublingual administration of the pharmaceutical tablet of claim 4 to a patient diagnosed with, or suspected of having, bronchial asthma.

45. A method for the treatment of a cardiac event, comprising the administration of a dose of the pharmaceutical tablet for buccal or sublingual administration of the pharmaceutical tablet of claim 4 to a patient diagnosed with, or suspected of having, a cardiac event.

46. A method for the treatment of a cardiac arrest, comprising the administration of a dose of the pharmaceutical tablet for buccal or sublingual administration of the pharmaceutical tablet of claim 4 to a patient diagnosed with, or suspected of having, a cardiac arrest
